SEQUENCE LISTING (42 sequences)

SEQ ID NO: 1 - protein, 32 aa, Homo sapiens
  DISULFID 10..26: note = Intrachain disulfide bond
SPKMVQGSGCFGRKMDRISSSSGLGCKVLRRH

SEQ ID NO: 2 - protein, 20 aa, synthetic construct
ASPAAPAPASPAAPAPSAPA

SEQ ID NO: 3 - protein, 20 aa, synthetic construct
AAPASPAPAAPSAPAPAAPS

SEQ ID NO: 4 - protein, 20 aa, synthetic construct
APSSPSPSAPSSPSPASPSS

SEQ ID NO: 5 - protein, 20 aa, synthetic construct
SAPSSPSPSAPSSPSPASPS

SEQ ID NO: 6 - protein, 20 aa, synthetic construct
SSPSAPSPSSPASPSPSSPA

SEQ ID NO: 7 - protein, 24 aa, synthetic construct
AASPAAPSAPPAAASPAAPSAPPA

SEQ ID NO: 8 - protein, 20 aa, synthetic construct
ASAAAPAAASAAASAPSAAA

SEQ ID NO: 9 - protein, 17 aa, synthetic construct
APAAPAPAPAAPAPAPA

SEQ ID NO: 10 - protein, 17 aa, synthetic construct
AAPAPAPAAPAPAPAAP

SEQ ID NO: 11 - protein, 10 aa, synthetic construct
APPPAPPPAP

SEQ ID NO: 12 - protein, 10 aa, synthetic construct
PAPPPAPPPA

SEQ ID NO: 13 - protein, 20 aa, synthetic construct
AAPAAPAPPAAAPAAPAPPA

SEQ ID NO: 14 - protein, 16 aa, synthetic construct
AAAAPAAAAAAAPAAA

SEQ ID NO: 15 - protein, 32 aa, synthetic construct
  DISULFID 10..26: note = Intrachain disulfide bond
CPKMVQGSGCFGRKMDRISSSSGLGCKVLRRH

SEQ ID NO: 16 - protein, 32 aa, synthetic construct
  DISULFID 10..26: note = Intrachain disulfide bond
SPCMVQGSGCFGRKMDRISSSSGLGCKVLRRH

SEQ ID NO: 17 - protein, 32 aa, synthetic construct
  DISULFID 10..26: note = Intrachain disulfide bond
SPKCVQGSGCFGRKMDRISSSSGLGCKVLRRH

SEQ ID NO: 18 - protein, 32 aa, synthetic construct
  DISULFID 10..26: note = Intrachain disulfide bond
SPKMCQGSGCFGRKMDRISSSSGLGCKVLRRH

SEQ ID NO: 19 - protein, 32 aa, synthetic construct
  DISULFID 10..26: note = Intrachain disulfide bond
SPKMVCGSGCFGRKMDRISSSSGLGCKVLRRH

SEQ ID NO: 20 - protein, 32 aa, synthetic construct
  DISULFID 10..26: note = Intrachain disulfide bond
SPKMVQGCGCFGRKMDRISSSSGLGCKVLRRH

SEQ ID NO: 21 - DNA, 157 bp, synthetic construct
  source 1..157: mol_type = other DNA
aggtaacatatgcctgccagaagagctcctcagcgctcttctgccagtccgaaaatggttcaaggtagcggttgttttggtcgtaaaatggatcgtattagcagcagcagcggtctgggttgtaaagttctgcgtcgtcattaataagcttgggttg

SEQ ID NO: 22 - DNA, 151 bp, synthetic construct
  source 1..151: mol_type = other DNA
aggtaacatatgccagccagaagagctcctcagcgctcttctgccaaaatggttcaaggtagcggttgttttggtcgtaaaatggatcgtattagcagcagcagcggtctgggttgtaaagttctgcgtcgtcattaataagcttgggttg

SEQ ID NO: 23 - DNA, 150 bp, synthetic construct
  source 1..150: mol_type = other DNA
attcgttcaggtaacatatgccagccagaagagctcctcagcgctcttctgcccaaggtagcggttgttttggtcgtaaaatggatcgtattagcagcagcagcggtctgggttgtaaagttctgcgtcgtcattaataagcttgggttg

SEQ ID NO: 24 - DNA, 154 bp, synthetic construct
  source 1..154: mol_type = other DNA
aggtaacatatgagtccgaaaatggttcaaggtagcggttgttttggtcgtaaaatggatcgtattagcagcagcagcggtctgggttgtaaagttctgcgtcgtcatgccagaagagctcctcagcgctcttctgcctaataagcttgggttg

SEQ ID NO: 25 - DNA, 148 bp, synthetic construct
  source 1..148: mol_type = other DNA
aggtaacatatgagtccgaaaatggttcaaggtagcggttgttttggtcgtaaaatggatcgtattagcagcagcagcggtctgggttgtaaagttctgcgtgccagaagagctcctcagcgctcttctgcctaataagcttgggttg

SEQ ID NO: 26 - DNA, 603 bp, synthetic construct
  source 1..603: mol_type = other DNA
gccagccctgccgcacctgcgcccgcatcacctgcggcacctgcaccttccgccccggctgcatctcctgccgcacccgcgcctgccagcccagctgcacctgccccaagtgcgccagcagcatcccctgccgcgcctgcccccgctagtccagcggccccagctccatctgcaccagctgctagccctgctgcaccagctcctgcttctcccgcagccccagcgccttctgctcccgcagcctcacctgcggccccggcaccagcatctccagcggcaccagcaccttcggcccctgctgctagcccagcagcacctgcgccagcctcaccagctgctcccgctcctagtgccccggcggcctcgcctgctgctcctgcaccagcttcgccagcggcaccggctccttcggcgccggctgcttcaccagcagcacctgctccagcgtccccagcggcccctgctccaagtgctccggctgcatcgcctgccgctcctgctcctgcatccccagctgctccagcaccaagcgcacctgccgcctcaccagcggcgccagcacccgccagcccagcagcgcctgctccatccgcaccggcggcc

SEQ ID NO: 27 - DNA, 4674 bp, synthetic construct
  source 1..4674: mol_type = other DNA
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttccgcgaaattaatacgactcactataggggaattgtgagcggataacaattcccctctagaaataattttgtttaactttttgagaccttaaggaggtaaaacatatgcctgccagccctgccgcacctgcgcccgcatcacctgcggcacctgcaccttccgccccggctgcatctcctgccgcacccgcgcctgccagcccagctgcacctgccccaagtgcgccagcagcatcccctgccgcgcctgcccccgctagtccagcggccccagctccatctgcaccagctgctagccctgctgcaccagctcctgcttctcccgcagccccagcgccttctgctcccgcagcctcacctgcggccccggcaccagcatctccagcggcaccagcaccttcggcccctgctgctagcccagcagcacctgcgccagcctcaccagctgctcccgctcctagtgccccggcggcctcgcctgctgctcctgcaccagcttcgccagcggcaccggctccttcggcgccggctgcttcaccagcagcacctgctccagcgtccccagcggcccctgctccaagtgctccggctgcatcgcctgccgctcctgctcctgcatccccagctgctccagcaccaagcgcacctgccgcctcaccagcggcgccagcacccgccagcccagcagcgcctgctccatccgcaccggcggccagtccgaaaatggttcaaggtagcggttgttttggtcgtaaaatggatcgtattagcagcagcagcggtctgggttgtaaagttctgcgtcgtcattaataagcttggttgaggtctcaccccctagcataaccccttggggcctctaaacgggtcttgaggggttttttgcccctgagacgcgtcaatcgagttcgtacctaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgactcatgaccaaaatcccttaacgtgagttacgcgcgcgtcgttccactgagcgtcagac

SEQ ID NO: 28 - DNA, 4668 bp, synthetic construct
  source 1..4668: mol_type = other DNA
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttccgcgaaattaatacgactcactataggggaattgtgagcggataacaattcccctctagaaataattttgtttaactttttgagaccttaaggaggtaaaacatatgccagccagccctgccgcacctgcgcccgcatcacctgcggcacctgcaccttccgccccggctgcatctcctgccgcacccgcgcctgccagcccagctgcacctgccccaagtgcgccagcagcatcccctgccgcgcctgcccccgctagtccagcggccccagctccatctgcaccagctgctagccctgctgcaccagctcctgcttctcccgcagccccagcgccttctgctcccgcagcctcacctgcggccccggcaccagcatctccagcggcaccagcaccttcggcccctgctgctagcccagcagcacctgcgccagcctcaccagctgctcccgctcctagtgccccggcggcctcgcctgctgctcctgcaccagcttcgccagcggcaccggctccttcggcgccggctgcttcaccagcagcacctgctccagcgtccccagcggcccctgctccaagtgctccggctgcatcgcctgccgctcctgctcctgcatccccagctgctccagcaccaagcgcacctgccgcctcaccagcggcgccagcacccgccagcccagcagcgcctgctccatccgcaccggcggccaaaatggttcaaggtagcggttgttttggtcgtaaaatggatcgtattagcagcagcagcggtctgggttgtaaagttctgcgtcgtcattaataagcttggttgaggtctcaccccctagcataaccccttggggcctctaaacgggtcttgaggggttttttgcccctgagacgcgtcaatcgagttcgtacctaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgactcatgaccaaaatcccttaacgtgagttacgcgcgcgtcgttccactgagcgtcagac

SEQ ID NO: 29 - DNA, 4659 bp, synthetic construct
  source 1..4659: mol_type = other DNA
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttccgcgaaattaatacgactcactataggggaattgtgagcggataacaattcccctctagaaataattttgtttaactttttgagaccttaaggaggtaaaacatatgccagccagccctgccgcacctgcgcccgcatcacctgcggcacctgcaccttccgccccggctgcatctcctgccgcacccgcgcctgccagcccagctgcacctgccccaagtgcgccagcagcatcccctgccgcgcctgcccccgctagtccagcggccccagctccatctgcaccagctgctagccctgctgcaccagctcctgcttctcccgcagccccagcgccttctgctcccgcagcctcacctgcggccccggcaccagcatctccagcggcaccagcaccttcggcccctgctgctagcccagcagcacctgcgccagcctcaccagctgctcccgctcctagtgccccggcggcctcgcctgctgctcctgcaccagcttcgccagcggcaccggctccttcggcgccggctgcttcaccagcagcacctgctccagcgtccccagcggcccctgctccaagtgctccggctgcatcgcctgccgctcctgctcctgcatccccagctgctccagcaccaagcgcacctgccgcctcaccagcggcgccagcacccgccagcccagcagcgcctgctccatccgcaccggcggcccaaggtagcggttgttttggtcgtaaaatggatcgtattagcagcagcagcggtctgggttgtaaagttctgcgtcgtcattaataagcttggttgaggtctcaccccctagcataaccccttggggcctctaaacgggtcttgaggggttttttgcccctgagacgcgtcaatcgagttcgtacctaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgactcatgaccaaaatcccttaacgtgagttacgcgcgcgtcgttccactgagcgtcagac

SEQ ID NO: 30 - DNA, 4671 bp, synthetic construct
  source 1..4671: mol_type = other DNA
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttccgcgaaattaatacgactcactataggggaattgtgagcggataacaattcccctctagaaataattttgtttaactttttgagaccttaaggaggtaaaacatatgagtccgaaaatggttcaaggtagcggttgttttggtcgtaaaatggatcgtattagcagcagcagcggtctgggttgtaaagttctgcgtcgtcatgccagccctgccgcacctgcgcccgcatcacctgcggcacctgcaccttccgccccggctgcatctcctgccgcacccgcgcctgccagcccagctgcacctgccccaagtgcgccagcagcatcccctgccgcgcctgcccccgctagtccagcggccccagctccatctgcaccagctgctagccctgctgcaccagctcctgcttctcccgcagccccagcgccttctgctcccgcagcctcacctgcggccccggcaccagcatctccagcggcaccagcaccttcggcccctgctgctagcccagcagcacctgcgccagcctcaccagctgctcccgctcctagtgccccggcggcctcgcctgctgctcctgcaccagcttcgccagcggcaccggctccttcggcgccggctgcttcaccagcagcacctgctccagcgtccccagcggcccctgctccaagtgctccggctgcatcgcctgccgctcctgctcctgcatccccagctgctccagcaccaagcgcacctgccgcctcaccagcggcgccagcacccgccagcccagcagcgcctgctccatccgcaccggcggcctaataagcttggttgaggtctcaccccctagcataaccccttggggcctctaaacgggtcttgaggggttttttgcccctgagacgcgtcaatcgagttcgtacctaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgactcatgaccaaaatcccttaacgtgagttacgcgcgcgtcgttccactgagcgtcagac

SEQ ID NO: 31 - DNA, 4665 bp, synthetic construct
  source 1..4665: mol_type = other DNA
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttccgcgaaattaatacgactcactataggggaattgtgagcggataacaattcccctctagaaataattttgtttaactttttgagaccttaaggaggtaaaacatatgagtccgaaaatggttcaaggtagcggttgttttggtcgtaaaatggatcgtattagcagcagcagcggtctgggttgtaaagttctgcgtgccagccctgccgcacctgcgcccgcatcacctgcggcacctgcaccttccgccccggctgcatctcctgccgcacccgcgcctgccagcccagctgcacctgccccaagtgcgccagcagcatcccctgccgcgcctgcccccgctagtccagcggccccagctccatctgcaccagctgctagccctgctgcaccagctcctgcttctcccgcagccccagcgccttctgctcccgcagcctcacctgcggccccggcaccagcatctccagcggcaccagcaccttcggcccctgctgctagcccagcagcacctgcgccagcctcaccagctgctcccgctcctagtgccccggcggcctcgcctgctgctcctgcaccagcttcgccagcggcaccggctccttcggcgccggctgcttcaccagcagcacctgctccagcgtccccagcggcccctgctccaagtgctccggctgcatcgcctgccgctcctgctcctgcatccccagctgctccagcaccaagcgcacctgccgcctcaccagcggcgccagcacccgccagcccagcagcgcctgctccatccgcaccggcggcctaataagcttggttgaggtctcaccccctagcataaccccttggggcctctaaacgggtcttgaggggttttttgcccctgagacgcgtcaatcgagttcgtacctaagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgactcatgaccaaaatcccttaacgtgagttacgcgcgcgtcgttccactgagcgtcagac

SEQ ID NO: 32 - protein, 234 aa, synthetic construct
PASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPKMVQGSGCFGRKMDRISSSSGLGCKVLRRH

SEQ ID NO: 33 - protein, 434 aa, synthetic construct
PASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPKMVQGSGCFGRKMDRISSSSGLGCKVLRRH

SEQ ID NO: 34 - protein, 634 aa, synthetic construct
PASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPKMVQGSGCFGRKMDRISSSSGLGCKVLRRH

SEQ ID NO: 35 - protein, 834 aa, synthetic construct
PASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPAAPAPASPAAPAPSAPAASPKMVQGSGCFGRKMDRISSSSGLGCKVLRRH

SEQ ID NO: 36 - protein, 32 aa, Homo sapiens; identical to SEQ ID NO: 1

SEQ ID NO: 37 - protein, 32 aa, synthetic construct; identical to SEQ ID NO: 15

SEQ ID NO: 38 - protein, 32 aa, synthetic construct; identical to SEQ ID NO: 16

SEQ ID NO: 39 - protein, 32 aa, synthetic construct; identical to SEQ ID NO: 17

SEQ ID NO: 40 - protein, 32 aa, synthetic construct; identical to SEQ ID NO: 18

SEQ ID NO: 41 - protein, 32 aa, synthetic construct; identical to SEQ ID NO: 19

SEQ ID NO: 42 - protein, 32 aa, synthetic construct; identical to SEQ ID NO: 20

(19) United States
(12) Patent Application Publication
(10) Pub. No.: US 2024/0199716 A1
(43) Pub. Date: Jun. 20, 2024

(54) MODIFIED B-TYPE NATRIURETIC PEPTIDE

(71) Applicant: Antlia BioScience, Inc., Encinitas, CA (US)

(72) Inventors: Nigel Shankley, Gunnislake (GB); Brian Johnson, Encinitas, CA (US); Sarkis Barret Kalindjian, Banstead (GB); Lars Friedrich, Munich (DE)

(73) Assignee: Antlia BioScience, Inc., Encinitas, CA (US)

(21) Appl. No.: 18/509,195

(22) Filed: Nov. 14, 2023

Related U.S. Application Data
(63) Continuation-in-part of application No. PCT/US22/29436, filed on May 16, 2022.
(60) Provisional application No. 63/188,743, filed on May 14, 2021.

(51) Int. Cl. C07K 14/575; A61K 38/00; C07K 14/00

(52) U.S. Cl. CPC C07K 14/575; C07K 14/001; A61K 38/00

(57) ABSTRACT

Provided is a modified B-type natriuretic peptide (BNP) comprising a covalently attached polymer comprising amino acids, where the polymer inhibits degradation and/or elimination of the BNP in a subject, and where the modified BNP retains vasorelaxant activity. Nucleic acid molecules encoding the above-described modified BNP, as are vectors comprising the nucleic acid molecules, and cells comprising the vector. Methods of treating a subject suffering from or diagnosed with a disease, disorder, or medical condition that can be treated with a natriuretic, diuretic or vasorelaxant is also provided. The methods comprise administering the above modified BNP to the subject. Further provided is method of preparing the above-described modified BNP. Additionally provided is the use of the above-described modified BNP, the above-described nucleic acid, the above-described vector, and/or the above-described cell for the manufacture of a medicament for the treatment of a disease, disorder, or medical condition that can be treated with a natriuretic, diuretic or vasorelaxant.

### CROSS-REFERENCE TO RELATED APPLICATION

This is a continuation-in-part of International Application No. PCT/US2022/029436, filed May 16, 2022, which claims the benefit of U.S. Provisional Application No. 63/188,743, filed May 14, 2021, both of which are incorporated by reference herein in its entirety.

### SEQUENCE LISTING

The instant application contains a Sequence Listing which has been submitted electronically in XML format and is hereby incorporated by reference in its entirety. Said XML copy, created on Jan. 17, 2024, is named ANTL-04-CIP_SL.xml and is 67,251 bytes in size.

### BACKGROUND OF THE INVENTION

### (1) Field of the Invention

The present application generally relates to therapeutic peptides. More specifically, the invention is directed to modified B-type natriuretic peptide (BNP) having decreased degradation and/or elimination in mammals.

### (2) Description of the Related Art

B-type natriuretic peptide (also known as Brain Natriuretic Peptide (or “BNP”) and sold as a commercial product named nesiritide and NATRECOR®), is an endogenous peptide belonging to the group of natriuretic peptides. BNP is a 32 amino acid peptide and was originally discovered in extract of porcine brain, leading to the name brain natriuretic peptide. A description of the protein is provided as the mature protein listed in NCBI Reference Sequence NP_002512.1 (“natriuretic peptides B preproprotein [Homo sapiens]”):

(SEQ ID NO: 36)
SPKMVQGSGC FGRKMDRISS SSGLGCKVLR RH

It is present in human brain, but there are significantly higher amounts in the cardiac ventricular tissue. BNP is released as a response to increased myocardial wall stretch, which is exaggerated in heart failure and is therefore used as a marker for pathology related to high extracellular fluid volumes.

Therapeutic measures related to diseases associated with sodium and water retention are varied and include administration of a variety of diuretic substances. BNP has natriuretic, diuretic, vasorelaxant, broncho-dilatory effects and may have antagonistic effects on the renin-angiotensin-aldosterone system. It is understood that these peptides and their analogs (such as atrial natriuretic peptide (ANP), BNP, C-type natriuretic peptide (CNP) and urodilatin (Uro) are effective in regulating blood pressure by controlling fluid volume and blood vessel diameter. In addition these peptides produce anti-fibrotic and anti-inflammatory effects.

Several disease states are characterized by abnormal fluid retention, including congestive heart failure, cirrhosis of the liver and nephrotic syndrome. These diseases are associated with excessive fluid accumulation on the venous side of circulation, and an under-perfusion of the kidneys, leading to a fall in glomerular filtration rate (GFR). Since 1980, the following advances have occurred where: BNP is cloned and expressed; and a commercial product named nesiritide (or NATRECOR®) has been approved by FDA for clinical indications of management of acute decompensated congestive heart failure (ADHF). Nesiritide and related medical uses are described in U.S. Pat. Nos. 5,114,923, 5,674,710, 6,586,396, 6,974,861, and 7,179,790. There are problems associated with the administration of nesiritide (see, e.g., O'Connor, 2011), including a short half-life in a human subject, and the product has not been regulated to treat chronic heart failure or other cardiovascular, metabolic, renal or pulmonary diseases other than ADHF.

More recently, a PEGylated BNP product described in Pub. No. WO2009156481A1 is prepared in anticipation of treating chronic heart failure which reaches peak level in plasma concentration between 2-4 hours of continuous transfusion. The PEGylated BNP described in that application is also immunogenic which causes problems with administration.

Therefore, a modified BNP is needed that has a longer duration blood level and is also less immunogenic than PEGylated BNP. The present invention addresses that need.

### BRIEF SUMMARY OF THE INVENTION

Provided is a modified B-type natriuretic peptide (BNP) comprising a sequence having at least 80%, at least 85%, at least 90%, at least 95%, at least 98% or 100% amino acid sequence identity to SEQ ID NO: 1. In these embodiments the modified BNP further comprises a covalently attached polymer comprising amino acids, where the polymer inhibits degradation and/or elimination of the BNP in a subject, and where the modified BNP retains vasorelaxant activity.

Also provided is a nucleic acid molecule encoding the above-described modified BNP.

Additionally provided is a vector comprising the above-described nucleic acid molecule.

Further provided is a cell comprising the above-described vector.

A method of treating a subject suffering from or diagnosed with a disease, disorder, or medical condition that can be treated with a natriuretic, diuretic or vasorelaxant is also provided. The method comprises administering to a subject in need of such treatment a therapeutically effective amount of the modified BNP described above.

Further provided is a method of preparing the above-described modified BNP. The method comprises expressing a modified BNP from the above-described cell as a fusion protein including the polymer or, alternatively, producing the BNP by solution or solid phase techniques and then covalently attaching a polymer using chemical methods.

Additionally provided is the use of the above-described modified BNP, the above-described nucleic acid, the above-described vector, and/or the above-described cell for the manufacture of a medicament for the treatment of a disease, disorder, or medical condition that can be treated with a natriuretic, diuretic or vasorelaxant.

### BRIEF DESCRIPTION OF THE SEVERAL VIEWS OF THE DRAWINGS

Those of skill in the art will understand that the drawings, described below, are for illustrative purposes only. The drawings are not intended to limit the scope of the present teachings in any way.

FIG. 1 is an illustration of the structure of native BNP (SEQ ID NO:1).

FIG. 2 is an illustration of a generalized depiction of native BNP bound to its receptor. Figure discloses SEQ ID NO: 1.

FIG. 3 is an illustration of sites of interest of native BNP related to its enzymatic degradation. Figure discloses SEQ ID NO: 1.

FIG. 4 is an illustration of sites of interest for BNP derivatization, including PASylation. Left to right and top to bottom—SEQ ID NOs: 15, 16, 17, 18, 19, 20.

FIG. 5 is an illustration of a PASylated BNP and method of manufacture.

FIG. 6 is a graph showing the results of an assay showing activation of human natriuretic polypeptide receptor (hNPR1) by BNP and BNP derivatives.

FIG. 7 is a graph showing relaxation of pre-contracted guinea pig tracheal ring segments by BNP and BNP derivative Compound 1.

FIG. 8 is a graph showing the two-phase model fit of canine plasma concentrations of Compound 1 over time following intravenous bolus dosing (0.2 mg/kg).

FIG. 9 is a graph showing the model fit of canine plasma concentrations of Compound 1 over time following subcutaneous bolus dosing (0.9 mg/kg).

FIG. 10 is graphs showing 24-hour post-dose telemetric recording of systolic blood pressure (SBP), diastolic blood pressure (DBP) and calculated mean arterial pressure (MAP=DBP+[0.33+(HR×0.0012)]×[SBP]) in dogs.

FIG. 11 is graphs showing 24-hour post-dose telemetric recording of mean arterial pressure (MAP) and heart rate (HR) following a subcutaneous bolus dose of 0.9 mg/kg Compound 1 or vehicle. Mean data (N=3).

FIG. 12 is a graph showing a 6-day recording of mean arterial pressure (MAP) following a subcutaneous bolus dose of 0.9 mg/kg Compound 1, an intravenous bolus dose of 0.2 mg/kg Compound 1 and a subcutaneous dose of vehicle (phosphate buffered saline). Mean data (N=3).

FIG. 13 is a graph showing a 5-day recording of mean arterial pressure (MAP) following a subcutaneous bolus dose of 0.9 mg/kg Compound 1, plotted on a reverse axis (right hand side) to allow visualization of the congruence with the plasma concentration of Compound 1.

FIG. 14 is a graph showing a 5-day recording of plasma cGMP concentration following a subcutaneous bolus dose of 0.9 mg/kg Compound 1, overlaid with corresponding plasma concentrations of Compound 1.

FIG. 15 is graphs illustrating the bioanalytical characterization of Compound 1 (PAS200) with size-exclusion chromatography (A) and ESI-mass spectroscopy (B).

FIG. 16 is a graph showing apparent potency and relative efficacy of human BNP1-32 and PAS analogues. Concentrations of cGMP in HEK293-T17 cells stimulated by hBNP(1-32), PAS200, PAS400 PAS600 and PAS800, were determined by an HTRF cGMP kit.

FIG. 17 is a graph showing plasma concentrations over time following a single intravenous bolus dosing of PAS200, PAS400, PAS600, and PAS800 in rats (N=4+/−SEM).

FIG. 18 is a graph showing the pharmacokinetic profiles of PAS200, PAS400, PAS600, and PAS800 following a single subcutaneous bolus dosing in rats (N=4+/−SEM).

### DETAILED DESCRIPTION OF THE INVENTION

### Abbreviations and Definitions

BNP: As used herein, the term “BNP” refers to B-type natriuretic peptide as described as the mature protein listed in NCBI Reference Sequence NP_002512.1 “natriuretic peptides B preproprotein [Homo sapiens]”.

BNP protein: By the terms “BNP protein” or “BNP peptide” or “BNP polypeptide” is meant an expression product of a BNP gene such as the native BNP protein, or a protein that shares at least 65% (but preferably 75%, 80%, 85%, 90%, 95%, 96%, 97%, 98%, or 99%) amino acid sequence identity with one of the foregoing and displays a functional activity of native BNP protein. The term can include derivatives of BNP which comprise a recombinant polypeptide covalently linked to one or both of the amino or carboxy terminal of the BNP protein. Such recombinant protein can be a BNP protein, including a PASylated BNP protein. The term can also include synthetic derivatives of BNP having a branched or unbranched polypeptide structure, for example where a polypeptide is covalently linked to one or more of the amino acids which comprise the BNP protein. In both the recombinant and synthetic aspects of the invention, the resulting polypeptide displays a biological activity of native BNP protein.

The terms “functional BNP protein” or “functional BNP” as used herein are intended to include a human BNP polypeptide having at least one functional activity of BNP.

Conservative changes: As used herein, when referring to mutations in a nucleic acid molecule, “conservative changes” are those in which at least one codon in the protein-coding region of the nucleic acid has been changed such that at least one amino acid of the polypeptide encoded by the nucleic acid sequence is substituted with another amino acid having similar characteristics. Examples of conservative amino acid substitutions are ser for ala, thr, or cys; lys for arg; gln for asn, his, or lys; his for asn; glu for asp or lys; asn for his or gln; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Functional activity: As used herein, the term “functional activity” refers to the biological effect of a substance on a living cell or organism. Accordingly, the terms “functional protein” or “functional peptide” or “functional polypeptide” as used herein relate to proteins or peptides or polypeptides that are capable of inducing, for example, a biological activity of BNP, e.g., its effectiveness in regulating blood pressure by controlling fluid volume and vessel diameter. In another example, a functional activity of a BNP protein can be identified as affecting abnormal fluid retention in certain tissues. Methods of determining a biological activity of BNP, as well as fragments, variants and homologs of BNP, are provided herein. Those of skill in the art will recognize other methods of measuring BNP activity, for example heart failure and fluid retention activity. Yet, it is of note that in the context of the present invention, the term “functional protein” relates to the whole protein of the invention which both comprises an amino acid sequence having and/or mediating said biological activity and an amino acid sequence forming random coil conformation, or other branched or unbranched derivatives of the BNP protein.

Accordingly, the terms “functional amino acid sequence” as used herein can relate to a “first domain” of the functional protein of the invention, mediating or having or being capable of mediating or having the above-defined biological activity. The terms “amino acid sequence having and/or mediating biological activity” or “amino acid sequence with biological activity” also relate to a “functional polypeptide” of the invention and relating to the “first domain” of said biologically active protein. Also comprised in the terms “amino acid sequence with functional activity” are functional fragments of BNP, the half-life of which, either in vivo or in vitro, is prolonged while at the same time reducing immunogenic activity. Accordingly, the proteins having functional activity in accordance with the present invention may comprise a functionally active amino acid sequence which is derived from naturally produced polypeptides or polypeptides produced by recombinant DNA technology.

Isolated polypeptide: The term “isolated polypeptide” as used herein means a polypeptide molecule is present in a form other than found in nature in its original environment with respect to its association with other molecules. The term “isolated polypeptide” encompasses a “purified polypeptide” which is used herein to mean that a specified polypeptide is in a substantially homogenous preparation, substantially free of other cellular components, other polypeptides, viral materials, or culture medium, or when the polypeptide is chemically synthesized, substantially free of chemical precursors or by-products associated with the chemical synthesis. A “purified polypeptide” can be obtained from natural or recombinant host cells by standard purification techniques, or by chemical synthesis.

The term “isolated polypeptide” also encompasses a “recombinant polypeptide,” which is used herein to mean a hybrid polypeptide produced by recombinant DNA technology or chemical synthesis having a specified polypeptide molecule covalently linked to one or more polypeptide molecules which do not naturally link to the specified polypeptide.

PASylation or PASylated: As used herein, the terms “PASylation” and “PASylated” are broadly defined to include BNP conjugated to conformationally disordered polymer sequences comprising the amino acids Pro, Ala, and, optionally, Ser (each a “PAS” group); Those of skill in the art will recognize that a PAS group may contain conservative substitutions, and the entire random coil comprising the Pro, Ala and optionally, Ser amino acids may also include conservative substituents. Hence, the term “PASylation” refers to attachment of a solvated random chain with large hydrodynamic volume to the BNP peptides. This amino acid string (polymer) adopts a bulky random coil structure, which significantly increases the size of the resulting modified peptide. By virtue of the significantly increased size of the modified peptide, typically rapid clearance of the biologically active component usually via kidney filtration is retarded by 1-2 orders of magnitude. Similarly, the bulk of the random coil structure may prevent the enzymatic degradation of the biologically active component.

Pharmaceutically acceptable: As used herein, the term “pharmaceutically acceptable” means approved by a regulatory agency of the Federal or a state government or listed in the U.S. Pharmacopeia or other generally recognized pharmacopeia for use in animals, and more particularly in humans.

Pharmaceutically acceptable carrier: As used herein, the term “pharmaceutically acceptable carrier” refers to a diluent, adjuvant, excipient, or vehicle with which a compound is administered. Such carriers can be sterile liquids, such as water and oils, including those of petroleum, animal, vegetable, or synthetic origin, such as peanut oil, soybean oil, mineral oil, sesame oil and the like, polyethylene glycols, glycerine, propylene glycol, or other synthetic solvents. Water is a preferred carrier when a compound is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. A compound, if desired, can also combine minor amount of wetting or emulsifying agents, or pH buffering agents such as acetates, citrates, or phosphates. Antibacterial agents such as a benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compounds in combination with carriers are known to those of skill in the art.

Pharmaceutically acceptable salt: As used herein, the term “pharmaceutically acceptable salt” includes those salts of a pharmaceutically acceptable compound formed with free amino groups such as those derived from hydrochloric, phosphoric, acetic, oxalic, and tartaric acids, and those formed with free carboxyl groups such as those derived from sodium, potassium, ammonium, calcium, ferric hydroxides, isopropylamine, triethylamine, 2-ethylamino ethanol, histidine, and procaine. If the compound is basic, salts may be prepared from pharmaceutically acceptable non-toxic acids including inorganic and organic acids. Such acids include acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic, and the like. Particularly preferred are besylate, hydrobromic, hydrochloric, phosphoric, and sulfuric acids. If the compound is acidic, salts may be prepared from pharmaceutically acceptable organic and inorganic bases. Suitable organic bases include, but are not limited to, lysine, N,N′-dibenzylethylenediamine, chloroprocaine, choline, diethanolamine, ethylene diamine, meglumine (N-methyl-glucamine) and procaine. Suitable inorganic bases include, but are not limited to, alkaline and earth-alkaline metals such as aluminum, calcium, lithium, magnesium, potassium, sodium, and zinc. Methods for synthesizing such salts are known to those of skill in the art.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein”, and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, mimetics (Mason, 2010) and the like. Modifications also include intra-molecular crosslinking and covalent attachment of various moieties such as lipids, flavin, biotin, polyethylene glycol or derivatives thereof, and the like. In addition, modifications may also include cyclization, branching and cross-linking. Further, amino acids other than the conventional twenty amino acids encoded by genes may also be included in a polypeptide.

The term “protein” or “polypeptide” may also encompass a “purified” polypeptide that is substantially separated from other polypeptides in a cell or organism in which the polypeptide naturally occurs (e.g., 30%, 40%, 50%, 60%, 70%, 80%, 90%, 95%, 96%, 97%, 98%, 99%, 100% free of contaminants).

The terms “primer,” “probe,” and “oligonucleotide” may be used herein interchangeably to refer to a relatively short nucleic acid fragment or sequence. They can be DNA, RNA, or a hybrid thereof, or chemically modified analogs or derivatives thereof. Typically, they are single-stranded. However, they can also be double-stranded having two complementing strands that can be separated by denaturation. In certain aspects, they are of a length of from about 8 nucleotides to about 200 nucleotides, preferably from about 12 nucleotides to about 100 nucleotides, and more preferably about 18 to about 50 nucleotides. They can be labeled with detectable markers or modified in any conventional manners for various molecular biological applications.

Random coil: As used herein, the term “random coil” relates to any conformation of a polymeric molecule, including amino acid polymers, in which the individual monomelic elements that form said polymeric structure are essentially randomly oriented towards the adjacent monomelic elements while still being chemically bound to said adjacent monomelic elements. In particular, a polypeptide or amino acid polymer adopting/having/forming “random coil conformation” substantially lacks a defined secondary and tertiary structure. The nature of polypeptide random coils and their methods of experimental identification are known to the person skilled in the art and have been described in the scientific literature (Cantor (1980) Biophysical Chemistry, 2nd ed., W. H. Freeman and Company, New York; Creighton (1993) Proteins—Structures and Molecular Properties, 2nd ed., W. H. Freeman and Company, New York; Smith (1996) Fold Des 1:R95-R106).

Therapeutically effective amount: As used herein, the term “therapeutically effective amount” refers to those amounts that, when administered to a particular subject in view of the nature and severity of that subject's disease or condition, will have a desired therapeutic effect, e.g. an amount that will cure, prevent, inhibit, or at least partially arrest or partially prevent a target disease or condition.

Transformed, transfected or transgenic: A cell, tissue, or organism into which has been introduced a foreign nucleic acid, such as a recombinant vector, is considered “transformed,” “transfected,” or “transgenic.” A “transgenic” or “transformed” cell or organism also includes progeny of the cell or organism, including progeny produced from a breeding program employing such a “transgenic” cell or organism as a parent in a cross.

Treatment: As used herein in the context of modified BNP, the terms “treat”, “treatment”, and the like, refer to relief from or alleviation of pathological processes mediated by modified BNP administration. In the context of the present invention insofar as it relates to any of the other conditions recited herein below, the terms “treat”, “treatment”, and the like mean to relieve or alleviate at least one symptom associated with such condition, or to slow or reverse the progression of such condition.

Vector: As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”.

Linker: The term “linker” refers to a short amino acid sequence that separates multiple domains of a polypeptide.

Methods involving conventional molecular biology techniques are generally known in the art and are described in detail in methodology treatises such as Green and Sambrook (2012).

### Modified B-Type Natriuretic Peptide (BNP)

Provided are BNP that further comprises amino acid polymers, for example polymers consisting of proline and alanine residues and optionally serine residues (PAS). Such compounds can be used in the treatment of fibrotic diseases, inflammatory diseases, chronic heart failure and other abnormal fluid retention indications including pulmonary diseases such as emphysema, asthma and COPD. In some embodiments, the PASylation adds a solvated random chain with large hydrodynamic volume to the native BNP protein. The addition of PAS polymers (PASylation) has been successfully utilized on the 94-amino acid peptide adnectin to increase plasma half-life (Aghaabdollahian, S. et al., 2019).

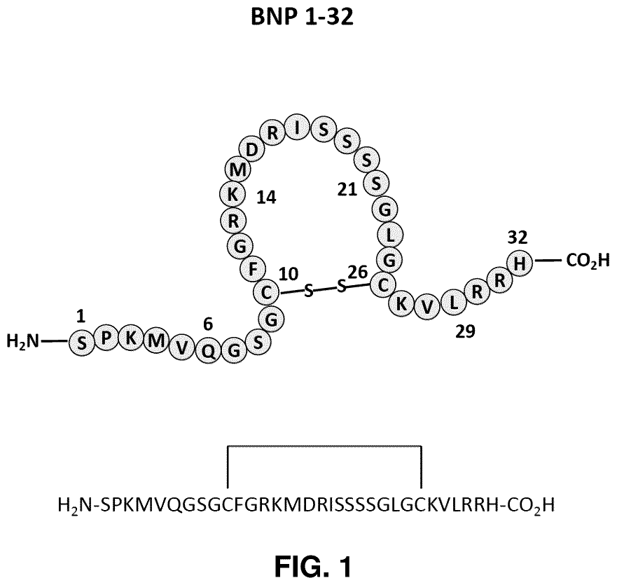

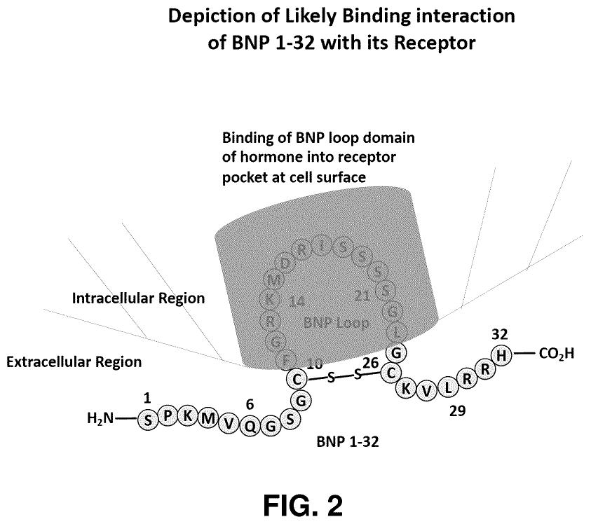

BNP 1-32 has cysteines at residues 10 and 26 which form a disulfide bridge, thus forming a loop structure in the middle section of the hormone. Extending from these residues are linear head (N-terminus) and tail (C-terminus) portions (FIG. 1).

There is crystallographic data available whereby a fragment of the molecule from the glycine at position 9, through the loop region to the leucine at position 29 is co-crystallized with a receptor protein closely analogous to the target receptor. This data suggests that this portion of the molecule is likely buried within the receptor structure, where there is little spare space. Further evidence for this comes from information derived from the whole 1-32 BNP molecule, whereby amphiphilic PEG oligomers attached to the lysines at positions 14 and 27 lost their agonist activities (Cataliotti et al., 2007).

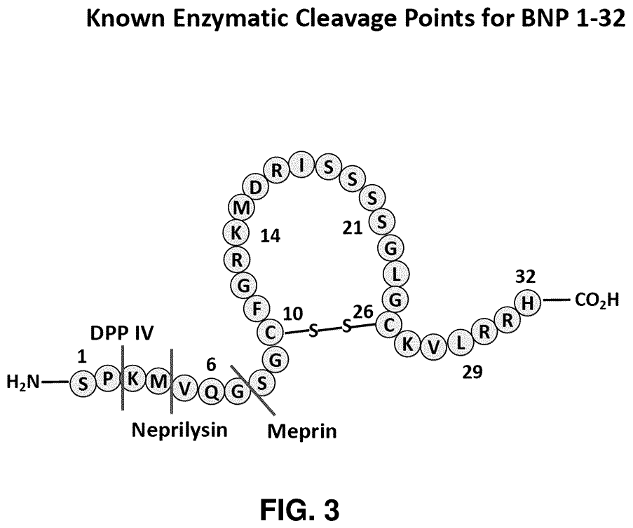

The hormone is processed by cleavage between residues 2 and 3 by the enzyme DPPIV, residues 4 and 5 by neprilysin and residues 7 and 8 by the metalloprotease, meprin. (FIG. 3). Where amphiphilic PEG oligomers are attached to the lysine at position 3, that activity is reasonably conserved and half-life extended. This suggests that some of the head portion of the BNP molecule, at least, is not involved in binding the receptor. Instead, the head portion of the BNP molecule is occupying a region where there is space for a macromolecule to be accommodated, presumably pointing away from the binding site of the receptor. The N-terminal region is therefore an advantageous part of the BNP molecule to modify, which contains processing points (e.g., cleavage points in FIG. 3) and residues probably not involved in binding to the receptor. However, the present invention encompasses PASylation of any point in the BNP molecule.

Thus, in some embodiments, a modified B-type natriuretic peptide (BNP) is provided. The modified BNP comprises a sequence having at least 80%, at least 85%, at least 90%, at least 95%, at least 98% or 100% amino acid sequence identity to SEQ ID NO: 1 or a portion thereof. In these embodiments, the modified BNP further comprises a covalently attached polymer comprising amino acids, wherein the polymer inhibits degradation and/or elimination of the BNP in a subject, and wherein the modified BNP retains vasorelaxant activity.

In some embodiments, the modified BNP has an altered sequence. These BNP protein variants such as fragments, analogs and derivatives of native BNP proteins are also within the invention. Such variants include, e.g., a polypeptide encoded by a naturally occurring allelic variant of a native BNP gene, a polypeptide encoded by an alternative splice form of a native BNP gene, a polypeptide encoded by a homolog of a native BNP gene, and a polypeptide encoded by a non-naturally occurring variant of a native BNP gene.

BNP protein variants have a peptide sequence that differs from a native BNP protein in one or more amino acids. The peptide sequence of such variants can feature a deletion, addition, or substitution of one or more amino acids of a native BNP polypeptide. Amino acid insertions can be about 1, 2, 3, 4, 5, 6, 7, 8, and 9 to 10 contiguous amino acids, and deletions can be about 1, 2, 3, 4, 5, 6, 7, 8, and 9 to 10 contiguous amino acids. In some applications, variant BNP proteins substantially maintain a BNP protein functional activity. For other applications, variant BNP proteins lack or feature a significant reduction in BNP protein functional activity. Where it is desired to retain a functional activity of native BNP protein, preferred BNP protein variants can be made by expressing nucleic acid molecules within the invention that feature silent or conservative changes. Variant BNP proteins with substantial changes in functional activity can be made by expressing nucleic acid molecules within the invention that feature less than conservative changes.

BNP protein fragments and variants corresponding to one or more particular motifs and/or domains or to arbitrary sizes, for example, at least 5, 6, 7, 8, 9, 10, 11, 12, 13, 14, 15, 16, 17, 18, 19, 20, 21, 22, 23, 24, 25, 26, 27, 28, 29, 30, 31, and 32 amino acids in length are intended to be within the scope of the present invention. Isolated peptidyl portions of BNP proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a BNP protein of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a native BNP protein.

Another aspect of the present invention concerns recombinant forms of the BNP proteins. Recombinant polypeptides preferred by the present invention, in addition to native BNP protein, are encoded by a nucleic acid that has at least 85% sequence identity (e.g., 85%, 86%, 87%, 88%, 89%, 90%, 91%, 92%, 93%, 94%, 95%, 96%, 97%, 98%, and 99%) with the nucleic acid sequence of NCBI Gene ID: 4879. In a preferred embodiment, variant BNP proteins have one or more functional activities of native BNP protein.

In various embodiments, the modified BNP is full length BNP. In other embodiments, the modified BNP is truncated at the C and/or N terminus of SEQ ID NO: 1. As shown in FIG. 1, BNP comprises a disulfide bridge between Cys 10 and Cys 26. BNP can be truncated towards the disulfide bridge from either the C or N terminus, or both, without substantial loss of activity. See, e.g., Example 4, where PASylated BNP1-30, PASylated BNP3-32 and PASylated BNP6-32 performed equivalent to PASylated BNP1-32 in an hNPR1 agonism assay.

The modified BNP can also include one or more additional proteins, either recombinantly or chemically attached covalently or noncovalently, for example one or more additional modified or unmodified BNP, a protein comprising an antibody binding site, a urocortin such as stresscopin, or any other bioactive protein.

Thus, the truncated BNP can be BNP2-32, BNP3-32, BNP4-32, BNP5-32, BNP6-32, BNP7-32, BNP8-32, BNP9-32, BNP10-32, BNP1-31, BNP2-31, BNP3-31, BNP4-31, BNP5-31, BNP6-31, BNP7-31, BNP8-31, BNP9-31, BNP10-31, BNP1-30, BNP2-30, BNP3-30, BNP4-30, BNP5-30, BNP6-30, BNP7-30, BNP8-30, BNP9-30, BNP10-30, BNP1-29, BNP2-29, BNP3-29, BNP4-29, BNP5-29, BNP6-29, BNP7-29, BNP8-29, BNP9-29, BNP10-29, BNP1-28, BNP2-28, BNP3-28, BNP4-28, BNP5-28, BNP6-28, BNP7-28, BNP8-28, BNP9-28, BNP10-28, BNP1-27, BNP2-27, BNP3-27, BNP4-27, BNP5-27, BNP6-27, BNP7-27, BNP8-27, BNP9-27, BNP10-27, BNP1-26, BNP2-26, BNP3-26, BNP4-26, BNP5-26, BNP6-26, BNP7-26, BNP8-26, or BNP9-26.

The polymers of these embodiments can be a variety of lengths and molecular weights. In some embodiments, the polypeptide forms a random coil structure. The polymers can have any length. In some embodiments, the polymer length is less than 200 amino acids. In other embodiments, the polymer length is 200 to 1000 amino acids, including in multiples of 200. Each 200 amino acid biopolymer unit confers a calculated molecular weight of about 17 kDa to the molecule to which it is attached. Use of modified amino acids or amino acid mimetics in these polymers are also envisioned.

The polymer of some of these modified BNP embodiments comprises amino acids consisting of proline and alanine residues and, optionally, serine residues (PAS).

In some embodiments, the polymer comprises any one or any combination of the following amino acid sequences:

(SEQ ID NO: 2)
ASPAAPAPASPAAPAPSAPA,
(SEQ ID NO: 3)
AAPASPAPAAPSAPAPAAPS,
(SEQ ID NO: 4)
APSSPSPSAPSSPSPASPSS,
(SEQ ID NO: 5)
SAPSSPSPSAPSSPSPASPS,
(SEQ ID NO: 6)
SSPSAPSPSSPASPSPSSPA,
(SEQ ID NO: 7)
AASPAAPSAPPAAASPAAPSAPPA,
(SEQ ID NO: 8)
ASAAAPAAASAAASAPSAAA,
(SEQ ID NO: 9)
APAAPAPAPAAPAPAPA,
(SEQ ID NO: 10)
AAPAPAPAAPAPAPAAP,
(SEQ ID NO: 11)
APPPAPPPAP,
(SEQ ID NO: 12)
PAPPPAPPPA,
(SEQ ID NO: 13)
AAPAAPAPPAAAPAAPAPPA
and
(SEQ ID NO: 14)
AAAAPAAAAAAAPAAA

or permuted or circular permuted versions or multimers(s) of these sequences as a whole or parts of these sequences.

It has been discovered that terminating the polymer with a proline aids in the subsequent purification of the modified BNP in some cases. Thus, in various embodiments, the polymer is terminated by a proline.

Particularly useful polymers comprise SEQ ID NO:2, repeated at least ten times, at least twenty times, at least thirty times, at least forty times or more, optionally terminated by a proline. In some of these embodiments, the polymer is bound to the BNP at an extra alanine of the polymer. In other embodiments the extra alanine is used to terminate the polymeric sequence.

The PAS polymer or polymers, of the modified BNP can be covalently bound to either or both of the N-terminus or the C-terminus of the BNP. Additionally, or alternatively, the polymer or polymers can be bound to any amino acid sidechain residue of the BNP outside of the disulfide bridge, i.e., any of residues 1, 2, 3, 4, 5, 6, 7, 8, 9, 27, 28, 29, 30, 31 or 32 of SEQ ID NO:1.

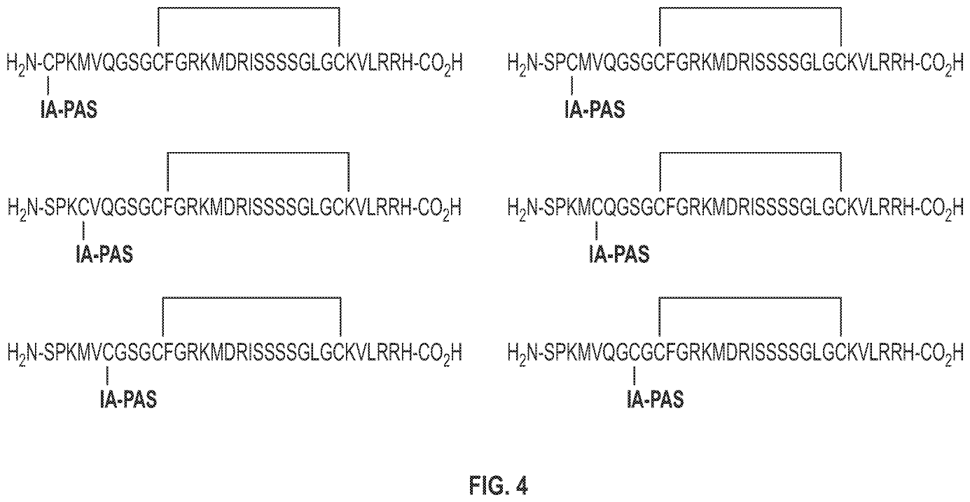

In some embodiments, the modified BNP comprises a cysteine inserted between, or substituting, any of residues 1-9 or 27-32 of SEQ ID NO:1. In these embodiments, the cysteine further comprises the polymer. See Example 1. FIG. 4 shows non-limiting examples of modified BNP where cysteine is substituted for the native amino acid in the BNP, and the PAS polymer is bound to the non-native cysteine. The amino acid polymer may be attached to a free cysteine in a BNP derivative using any of a variety of linkers known in the art including a methylcarbonyl group (IA) derived from an activated iodoacetic acid (IA in FIG. 4).

To facilitate conjugation of amino acid polymers to BNP, a linker between the BNP and the polymer may be utilized. Any linker known in the art that can facilitate this conjugation may be utilized. Examples are provided in US Patent Application Publication 2011/0105397, e.g., at para. 135, and references cited therein. In some embodiments, the linker moiety is N-(ethylcarbonyl)succinimide or methylcarbonyl. Those linkers have the structures

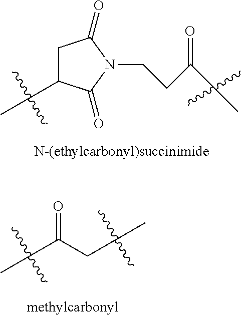 [SMILES: *C(=O)CCN1C(=O)CC(*)C1=O ; *CC(*)=O]

The length of the polymer in the modified BNP can be as long as 1200 amino acids, e.g., about 100, about 200, about 400, about 600, about 800, about 1000, or about 1200 amino acids, or any amount in between.

In some embodiments, a polymer is at the N or C terminus of the BNP. Such terminal polymers can be produced genetically, e.g., by coding the polymer with the BNP in a DNA sequence and expressing that sequence. Thus, a nucleic acid molecule encoding the modified BNP having an amino acid polymer at either or both of the N and/or C terminus is also provided herein, as is a vector comprising that nucleic acid molecule. A cell comprising that vector, including a cell capable of expressing that modified BNP is also provided herein.

Nonlimiting examples of specific modified BNPs provided herewith include P-(SEQ ID No:2)₁₀-A-hBNP(1-32) (PAS attached to N-terminal amino group) (Compound 1 in the Examples below), P-(SEQ ID No:2)₁₀-A-hBNP(3-32) (PAS attached to the alpha amino group of the N-Terminal lysine 3) (Compound 2 in the Examples below), P-(SEQ ID No:2)₁₀-A-hBNP(6-32) (PAS attached to N-terminus of glutamine 6) (Compound 3 in the Examples below), hBNP(1-32)-(SEQ ID No:2)₁₀-A (PAS attached to the C-terminus carboxy group) (Compound 4 in the Examples below), hBNP(1-30)-(SEQ ID No:2)₁₀-A (PAS attached to carboxy group of the C-Terminal arginine 30) (Compound 5 in the Examples below), P-(SEQ ID No:2)₂₀-A-hBNP(1-32) (PAS attached to N-terminal amino group), P-(SEQ ID No:2)₂₀-A-hBNP(3-32) (PAS attached to the alpha amino group of the N-Terminal lysine 3), P-(SEQ ID No:2)₂₀-A-hBNP(6-32) (PAS attached to N-terminus of glutamine 6), hBNP(1-32)-(SEQ ID No:2)₂₀-A (PAS attached to the C-terminus carboxy group), hBNP(1-30)-(SEQ ID No:2)₂₀-A (PAS attached to carboxy group of the C-Terminal arginine 30), P-(SEQ ID No:2)₃₀-A-hBNP(1-32) (PAS attached to N-terminal amino group), P-(SEQ ID No:2)₃₀-A-hBNP(3-32) (PAS attached to the alpha amino group of the N-Terminal lysine 3), P-(SEQ ID No:2)₃₀-A-hBNP(6-32) (PAS attached to N-terminus of glutamine 6), hBNP(1-32)-(SEQ ID No:2)₃₀-A (PAS attached to the C-terminus carboxy group), hBNP(1-30)-(SEQ ID No:2)₃₀-A (PAS attached to carboxy group of the C-Terminal arginine 30), P-(SEQ ID No:2)₄₀-A-hBNP(1-32) (PAS attached to N-terminal amino group), P-(SEQ ID No:2)₄₀-A-hBNP(3-32) (PAS attached to the alpha amino group of the N-Terminal lysine 3), P-(SEQ ID No:2)₄₀-A-hBNP(6-32) (PAS attached to N-terminus of glutamine 6), hBNP(1-32)-(SEQ ID No:2)₄₀-A (PAS attached to the C-terminus carboxy group), or hBNP(1-30)-(SEQ ID No:2)₄₀-A (PAS attached to carboxy group of the C-Terminal arginine 30). Nonlimiting examples of these modified BNPs comprise SEQ ID NO:32, SEQ ID NO:33, SEQ ID NO:34, or SEQ ID NO:35 (PAS200, PAS400, PAS600 or PAS800, as described in Example 7 below).

The inventors have discovered that the modified BNPs described above exhibit a surprisingly long half-life in mammals, for example when injected subcutaneously into a rat or dog (widely used and reliable model systems for determining half-life in mammals such as humans), See Examples 6 and 9 below.

Thus, in some embodiments, the modified BNPs exhibit a half-life greater than 8-fold, 10-fold, 15-fold, 20-fold, 30-fold, 40-fold, 50-fold, 60-fold, 70-fold, 80-fold, 90-fold, 100-fold, 150-fold, 200-fold, 250-fold, 300-fold or any value in between, of the native BNP when injected subcutaneously into a rat.

In some embodiments, the modified BNP is produced in the above cells. For example, a host cell transfected with a nucleic acid vector directing expression of a nucleotide sequence encoding the subject polypeptides can be cultured under appropriate conditions to allow expression of the peptide to occur. The cells may be harvested, lysed, and the protein isolated. A recombinant BNP protein can be isolated from host cells using techniques known in the art for purifying proteins including ion-exchange chromatography, gel filtration chromatography, ultrafiltration, electrophoresis, and immunoaffinity purification with antibodies specific for such protein.

### Pharmaceutical Preparations and Methods of Administration

In some embodiments, the modified BNP described above is formulated in a pharmaceutically acceptable carrier. Those compositions can be administered to a subject at therapeutically effective doses to treat any disease, disorder, or medical condition mediated by NPR1 activity. The subject can be any mammal, reptile or avian, including but not limited to horses, cows, dogs, cats, sheep, pigs, rats, mice, chickens, and humans.

### Therapeutically Effective Dosage

Toxicity and therapeutic efficacy of such compositions can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compositions that exhibit large therapeutic indices are preferred. While compositions exhibiting toxic side effects may be used, care should be taken to design a delivery system that targets such compositions to the site affected by the disease or disorder in order to minimize potential damage to unaffected cells and reduce side effects.

The data obtained from cell culture assays and animal studies can be used in formulating a range of dosages for use in humans and other mammals. The dosage of such compositions lies preferably within a range of circulating plasma or other bodily fluid concentrations that include the ED₅₀ with little or no toxicity. The dosage may vary within this range depending upon the dosage form employed and the route of administration utilized. For any composition of the invention, the therapeutically effective dose can be estimated initially from cell culture assays. A dosage may be formulated in animal models to achieve a circulating plasma concentration range that includes the EC₅₀ (the concentration of the test composition that achieves a half-maximal effect) as determined in cell culture. Such information can be used to more accurately determine useful dosages in humans and other mammals. Composition levels in plasma may be measured, for example, by high performance liquid chromatography.

The amount of a composition that may be combined with pharmaceutically acceptable carriers to produce a single dosage form will vary depending upon the host treated and the particular mode of administration. It will be appreciated by those skilled in the art that the unit content of a composition contained in an individual dose of each dosage form need not in itself constitute a therapeutically effective amount, as the necessary therapeutically effective amount could be reached by administration of a number of individual doses. The selection of dosage depends upon the dosage form utilized, the condition being treated, and the particular purpose to be achieved according to the determination of those skilled in the art.

The dosage regime for treating a disease or condition with the compositions and/or composition combinations of this invention is selected in accordance with a variety of factors, including the type, age, weight, sex, diet and medical condition of the patient, the route of administration, pharmacological considerations such as activity, efficacy, pharmacokinetic and toxicology profiles of the particular composition employed, whether a composition delivery system is utilized and whether the composition is administered as a pro-drug or part of a drug combination. Thus, the dosage regime actually employed may vary widely from subject to subject.

### Formulations and Use

The compositions of the present invention may be formulated by known methods for administration to a subject using several routes which include, but are not limited to, parenteral, oral, topical, intradermal, intramuscular, intraperitoneal, intravenous, subcutaneous, intranasal, epidural, inhaled and ophthalmic routes. The individual compositions may also be administered in combination with one or more additional compositions of the present invention and/or together with other biologically active or biologically inert agents (“composition combinations”). Such biologically active or inert agents may be in fluid or mechanical communication with the composition(s) or attached to the composition(s) by ionic, covalent, Van der Waals, hydrophobic, hydrophilic or other physical forces. It is preferred that administration is localized in a subject, but administration may also be systemic.

The compositions or composition combinations may be formulated by any conventional manner using one or more pharmaceutically acceptable carriers and/or excipients. Thus, the compositions and their pharmaceutically acceptable salts and solvates may be specifically formulated for administration, e.g., by parenteral, inhalation or insufflation (either through the mouth or the nose) or oral, buccal, parenteral or rectal administration. The composition or composition combinations may take the form of charged, neutral and/or other pharmaceutically acceptable salt forms. Examples of pharmaceutically acceptable carriers include, but are not limited to, those described in Remington's Pharmaceutical Sciences (A.R. Gennaro, Ed.), 20th edition, Williams & Wilkins PA, USA (2000).

The compositions may also take the form of solutions, suspensions, emulsions, tablets, pills, capsules, powders, controlled- or sustained-release formulations and the like. Such compositions will contain a therapeutically effective amount of the composition, preferably in purified form, together with a suitable amount of carrier so as to provide the form for proper administration to the patient. The formulation should suit the mode of administration.

### Parenteral Administration

The composition or composition combination may be formulated for parenteral administration by injection, e.g., by bolus injection or continuous infusion. Formulations for injection may be presented in unit dosage form in ampoules or in multi-dose containers with an optional preservative added. The parenteral preparation can be enclosed in ampoules, disposable syringes or multiple dose vials made of glass, plastic or the like. The composition may take such forms as suspensions, solutions or emulsions in oily or aqueous vehicles, and may contain formulatory agents such as suspending, stabilizing and/or dispersing agents.

For example, a parenteral preparation may be a sterile injectable solution or suspension in a nontoxic parenterally acceptable diluent or solvent (e.g., as a solution in 1,3-butanediol). Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid may be used in the parenteral preparation.

Alternatively, the composition may be in powder form for constitution with a suitable vehicle, such as sterile pyrogen-free water, before use. For example, a composition suitable for parenteral administration may comprise a sterile isotonic saline solution containing between 0.1 percent and 90 percent weight per volume of the composition or composition combination. By way of example, a solution may contain from about 5 percent to about 20 percent, more preferably from about 5 percent to about 17 percent, more preferably from about 8 to about 14 percent, and still more preferably about 10 percent of the composition. The solution or powder preparation may also include a solubilizing agent and a local anesthetic such as lignocaine to ease pain at the site of the injection. Other methods of parenteral delivery of compositions will be known to the skilled artisan and are within the scope of the invention.

### Other Systems of Administration

Various other delivery systems are known in the art and can be used to administer the compositions of the invention. Moreover, these and other delivery systems may be combined and/or modified to optimize the administration of the compositions of the present invention. In some embodiments, the formulation can be aerosolized.

### Active Ingredient Kits

In various embodiments, the present invention can also involve kits. Such kits can include the compositions of the present invention and, in certain embodiments, instructions for administration. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components. In addition, if more than one route of administration is intended or more than one schedule for administration is intended, the different components can be packaged separately and not mixed prior to use. In various embodiments, the different components can be packaged in one composition for administration together.

Kits may also include reagents in separate containers such as, for example, sterile water or saline to be added to a lyophilized active component packaged separately. For example, sealed glass ampules may contain lyophilized phosphatases and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a thumb drive, CD-ROM, DVD-ROM, video, audio, and the like. Detailed instructions may not be physically associated with the kit; instead, a user may be directed to an Internet web site specified by the manufacturer or distributor of the kit.

### Methods of Treatment

A method of treating a subject suffering from or diagnosed with a disease, disorder, or medical condition that can be treated with a natriuretic, diuretic or vasorelaxant, including fibrotic or inflammatory disease, is also provided. The method comprises administering to a subject in need of such treatment a therapeutically effective amount of any of the modified BNP described above.

In some embodiments, the disease, disorder, or medical condition is a hematological disease, a neurological disease, a developmental disease, a urological disease, a reproduction disorder, a psychiatric disorder, a cancer, an autoimmune disease, a fibrotic disease, an inflammatory disease, a neurodegenerative disease, an infectious disease, a lung disease, a heart disease, a vascular disease, or a metabolic disease.

In some of these embodiments, the disease, disorder, or medical condition is anxiety, depression, posttraumatic stress disorder, obesity, peripherally acting inflammatory bowel disease, irritable bowel syndrome, stress response, sleep disorder, addictive behavior, acute and chronic neurodegeneration, preterm labor or pain, vasculitis and/or excessive angiogenesis in an autoimmune disorder, systemic sclerosis, multiple sclerosis, Sjogren's disease, a vascular malformation in a blood and/or lymph vessel, left ventricular hypertrophy, portal vein hypertension, liver ascites, pulmonary hypertension, idiopathic pulmonary hypertension, atrial hypertension, chronic obstructive pulmonary disease, idiopathic pulmonary fibrosis, pulmonary fibrosis, DiGeorge syndrome, hereditary hemorrhagic telangiectasia, cavernous hemangioma, cutaneous hemangioma, a lymphatic malformation, transplant adenopathy, atherosclerosis, vascular anastomoses, adipose tissue in obesity, allograft rejection, a skin disease, psoriasis, warts, allergic dermatitis, scar keloids, pyogenic granulomas, blistering disease, Kaposi sarcoma in an AIDS patient, systemic sclerosis, an eye disease, persistent hyperplastic vitreous syndrome, diabetic retinopathy, retinopathy of prematurity, choroidal neovascularization, pulmonary hypertension, asthma, nasal polyps, rhinitis, chronic airway inflammation and obstruction, cystic fibrosis, acute lung injury, bronchiolitis obliterans organizing pneumonia, a gastrointestinal tract disease, inflammatory bowel disease, periodontal disease, ascites, peritoneal adhesions, liver cirrhosis, a reproductive system disease, endometriosis, uterine bleeding, ovarian cysts, ovarian hyperstimulation, a bone or joint disease, arthritis, synovitis, osteomyelitis, osteophyte formation, HIV-induced bone marrow angiogenesis, kidney disease, or early diabetic nephropathy.

As discussed above, the compositions can be administered by any appropriate method known in the art. In some embodiments, the administration is by injection. In other embodiments, the modified BNP is aerosolized and is administered by inhalation.

### Methods of Preparation

The above-described compositions can be prepared by any appropriate method known in the art. Where the polymer is at the N or C terminus of the BNP, the above-described cell comprising a vector encoding the modified BNP can express the modified BNP. Where the polymer is to be conjugated to one or more amino acid residues of BNP, the modified BNP can be produced by solution or solid phase techniques, then covalently attaching a polymer using chemical methods. Such techniques and methods are well-known in the art.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Utility of PASylated ANP and Urodilatin

The compositions and methods provided herein can be applied to ANP and urodilatin to make PASylated ANP and urodilatin that retain the biological activity of native ANP and urodilatin.

### EXAMPLES

### Example 1—BNP Derivative Synthesis

BNP 1-32 derivatives were obtained by solid phase or solution phase chemistry or a mixture of both. As a result of having two cysteines in the molecule that are required to form the disulfide bridge, as well as others to which to attach the PAS moiety, an orthogonal protecting group strategy was used. Other chemical synthesis techniques may be used to achieve the orthogonal protecting group strategy.

The PAS group itself is prepared by recombinant means. The recombinant product is isolated before being derivatised at its N-terminus, usually an alanine residue, with a linking reagent, capable of reacting with the free cysteine thiol in the BNP derivative. The linkers used are methylcarbonyl (IA in FIG. 4) or N-(ethylcarbonyl)succinimide. When IA is used as a linker reagents consisting of appropriate sequences of proline and alanine or proline, alanine and serine, H₂OC-(Pro/Ala)-Ala-NHCOCH₂I or H₂OC-(Pro/Ala/Ser)-Ala-NHCOCH₂I are prepared from PAS by reacting with a carboxy-activated iodoacetic acid. These PASylating reagents in turn are then reacted with the free cysteine thiol in the BNP derivative, to obtain the PASylated peptide. For example, residue 1 may be mutated from S to C; residue 3 may be mutated from K to C; residue 4 may be mutated from M to C; residue 5 may be mutated from V to C; residue 6 may be mutated from Q to C; and residue 8 may be mutated from S to C.

PASylation leads to (A) retarded kidney filtration of BNP, while: (B) establishing whether any sidechains in the N-terminus are not essential in conferring receptor affinity to the hormone and (C) suppressing proteolytic enzymatic cleavages, which readily extends the half-life.

Those of skill in the art will recognize that other methods of PASylating the BNP protein can be utilized, including via chemistry which modifies the C-terminus of the BNP protein or by heterologous gene expression of a BNP that is genetically fused either N- or C-terminally with a PAS sequence or polymer.

### Example 2—PASylation of BNP

Synthetic DNA fragments encoding the amino acids 1-32 (Compound 1 and 4), 3-32 (Compound 2), 6-32 (Compound 3) or 1-30 (Compound 5) of human BNP were obtained from Thermo Fisher Scientific (Regensburg, Germany). The gene fragments for Compounds 1 to 3 (SEQ ID NOs:21, 22, 23) comprised an NdeI restriction site, followed by a CCT proline codon, a GCC alanine codon, a first SapI recognition sequence GCTCTTC on the non-coding strand, an 8-nucleotide spacer, and a second SapI restriction sequence in reverse orientation, with its recognition sequence GCTCTTC on the coding strand, followed by a GCC alanine codon and the coding sequence for human BNP (or a fragment thereof), which was finally followed by a HindIII restriction site. The order of coding elements on the gene fragment for Compounds 4 and 5 (SEQ ID NOs:24, 25) was as follows: NdeI restriction site, the coding sequence for human BNP (or a fragment thereof), a GCC alanine codon, a first SapI recognition sequence GCTCTTC on the non-coding strand, an 8-nucleotide spacer, and a second SapI restriction sequence in reverse orientation with its recognition sequence GCTCTTC on the coding strand, followed by a GCC alanine codon and a TAA stop codon.

In order to clone the BNP DNA constructs on the expression plasmid pD451-SR (ATUM, Newark, CA), the original SapI cloning site on the vector was replaced by a sequence comprising an NdeI and a HindIII recognition site. To this end, the vector was digested with XbaI and StyI and its backbone was religated with a double-stranded pair of synthetic oligonucleotides comprising a ribosome-binding site (RBS), an NdeI site, a HindIII site and flanking sticky ends compatible with the XbaI and StyI sites.

The BNP gene fragments were then inserted into the modified pD451-SR vector via the restriction sites NdeI and HindIII according to standard procedures (Sambrook (2012) Molecular Cloning: A Laboratory Manual, Cold Spring Harbor Laboratory Press). Subsequently, each resulting plasmid was digested with SapI, which led to the liberation of a small (27 bp) DNA insert containing the pair of SapI recognition sites as part of the synthetic DNA fragments described above and a vector backbone with compatible 5′-GCC/5′-GGC sticky ends at the position directly either upstream of the encoded N-terminus of BNP (Compounds 1-3) or downstream of the C-terminus (Compounds 4 and 5). This strategy is ideally suited for insertion of the low repetitive nucleic acid molecules encoding a proline/alanine-rich amino acid repeat sequence. After isolation of said vector fragment using the Wizard gel extraction kit (Promega, Mannheim, Germany) and dephosphorylation with the thermosensitive alkaline phosphatase FastAP (Thermo Fisher Scientific, Waltham, MA) it was ligated with the previously cloned PAS #1.2(200) gene cassette (SEQ ID NO:26) carrying compatible overhangs as described (WO 2017/109087 A1). The resulting plasmids allow the bacterial expression of in frame fusion proteins comprising the PAS sequence fused either N-terminally or C-terminally with the biologically active BNP peptide (or fragment thereof).

Competent E. coli T7express cells (New England Biolabs, Ipswich, MA) were transformed with either one of the following expression plasmids: pD451-SR-PAS200-BNP32 for Compound 1 (SEQ ID NO:27), pD451-SR-PAS200-BNP(3-32) for Compound 2 (SEQ ID NO:28), pD451-SR-PAS200-BNP(6-32) for Compound 3) (SEQ ID NO:29), pD451-SR-BNP32-PAS200 for Compound 4) (SEQ ID NO:30), pD451-SR-BNP(1-30)-PAS200 for Compound 5) (SEQ ID NO:31). An Erlenmeyer flask containing 50 mL TB medium (Carl Roth, Karlruhe, Germany) supplemented with kanamycin (30 mg/l) was inoculated with a single colony for each of the transformations and incubated over night at 37° C. 10 ml of this pre-culture was used to inoculate a shake-flask containing 2 L TB medium (with 30 mg/l kanamycin). After incubation at 30° C. for 16 h and reaching an optical density (OD₆₀₀) of ˜3, recombinant gene expression was induced by adding 1 mM isopropyl β-D-1-thiogalactopyranoside (IPTG) to the culture. The E. coli cells were harvested 4 h after induction by centrifugation (6,000 rpm, 25 min, 4° C.), and the cell pellet (about 10 g per shake flask) was frozen at −20° C.

Cell lysis was performed after resuspending the pellets in 100 mM Tris/HCl pH 8.5 (4.4 vol.) by adding 0.15% (v/v) tergitol type 15-S-9 (Sigma-Aldrich, St. Louis, MO), hen egg-white lysozyme (4 mg per 10 g pellet; Sigma-Aldrich), Cyanase Nuclease (250 U per 10 g pellet; SERVA Electrophoresis, Heidelberg, Germany) and 40 μM MgCl₂. After incubating the lysis mixture for 2 h on ice, the soluble fraction was separated from cell debris by centrifugation (39,000×g, 1.5 h, 4° C.). The cleared supernatant was subjected to ammonium sulfate precipitation (30% saturation at RT) and the precipitate was resolubilized in 25 mM Na-borate buffer (pH 9.5) supplemented with 1 mM EDTA. Residual ammonium sulfate was removed by dialysis against the borate buffer.

The resulting protein extract was subjected to subtractive anion exchange chromatography on a Fractogel EMD TMAE (S) column (Merck, Darmstadt, Germany) and subsequent cation exchange chromatography (binding mode) on a Fractogel EMD SO₃⁻ (S) column (Merck). The PAS200-BNP fusion protein was eluted from this column by applying an NaCl gradient of 0-500 mM in the borate buffer (see above). The eluate fractions were analyzed by SDS-PAGE, pooled as appropriate and dialyzed against ultra-pure water. The salt-free PAS200-BNP32 was lyophilized, resulting in a yield of 5-10 mg per 2 l shake flask culture as determined gravimetrically.

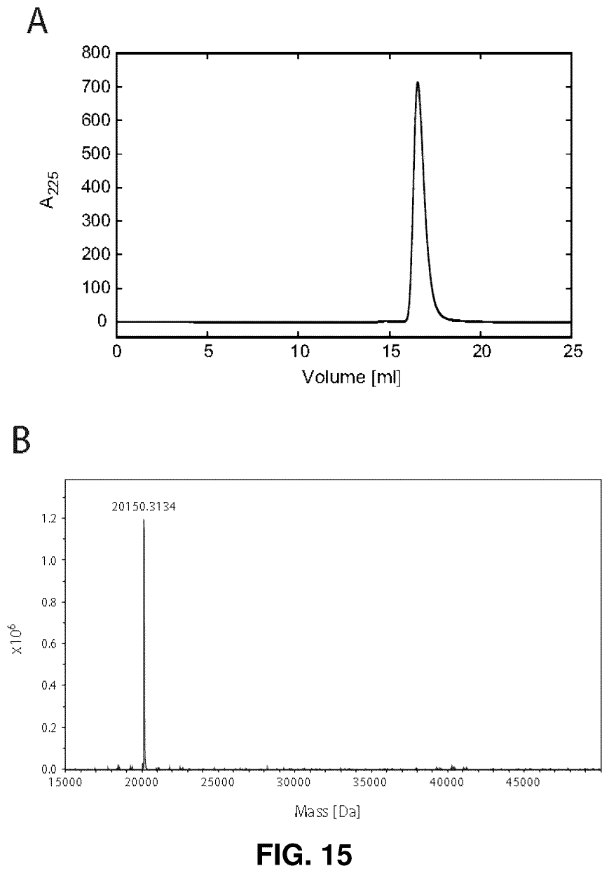

ESI-MS analysis (FIG. 15B) of the purified Compound 1 revealed a single mass peak corresponding to the expected mass of PAS200-BNP32 with the correctly formed intramolecular disulfide bridge (20150.7 Da), also indicating a cleaved start-Met. The mass spectrum did not reveal hints of potentially disulfide-linked PAS200-BNP dimers or signs of proteolytic degradation. In analytical size exclusion chromatography (SEC) on a Superose 6 increase 10/300 column (Cytiva, Uppsala, Sweden) with phosphate-buffered saline (PBS) as running buffer, the PASylated BNP eluted as a monodisperse macromolecule in a single peak at a volume of 16.5 ml (FIG. 15A), indicating a uniform polypeptide preparation. Calibration of the column with globular proteins of known molecular weights allowed the determination of an apparent molecular weight of 93 kDa for the PASylated BNP. This elevated apparent molecular weight is due to the random coil nature of the PAS moiety, which results in a strongly increased hydrodynamic volume.

### Example 3—Administration of BNP

### Study Objective

The study determines that long-term (3 month) treatment with BNP protein with induced random coiling in dogs with ischemia induced, progressive, irreversible heart failure is associated with: (1) preservation and/or improvement of LV structure and function; (2) no change in or long-term reduction in biomarkers of myocardial injury; and (3) absence of significant de-novo ventricular arrhythmias or increased susceptibility for malignant arrhythmias compared to placebo (vehicle). See also Example 6.

### Study Protocol

The study analyzes 24 dogs with advanced heart failure (HF) produced by multiple sequential intracoronary microembolizations (LV ejection fraction≤25%) (1). Dogs are randomized into 3 study groups. Group I (n=8) receives subcutaneous vehicle injection for 3 months, while serving as a placebo control. Group II (n=8) receives chronic therapy with BNP derivative (0.1 mg/kg, Q5d) for 3 months. Group III (n=8) receives chronic therapy with BNP derivative (0.3 mg/kg, Q5d) for 3 months. All dosing is performed at the same time of the day on a 20 cm×24 cm area: Within the 20 cm×24 cm area that is shaved on the anterior dorsal scapular region (scruff) of the animal's neck, six (6) regions are outlined with the center of each region 12 cm apart. The regions are numbered as outlined and where the order of injection is region 1, 5, 3, 6, 2, 4. Hemodynamic, angiographic and echocardiographic measurements are performed during a left and right heart catheterization under general anesthesia. A left and right heart catheterization are performed at baseline, 7 days prior to placebo vehicle or BNP derivative injection, 24 hrs following the first (1st) BNP derivative injection, 24 hrs following the third (3rd) BNP derivative injection (day 10), 24 hrs following the fifth (5th) BNP derivative injection (day 20), 24 hrs following the seventh (7th) BNP derivative injection (day 30), 24 hrs following the twelfth (12th) BNP derivative injection (day 60), and 24 hrs following the eighteenth (18th) BNP derivative injection (day 90). Following the hemodynamic and ventriculographic measurements on day 90, the chest is rapidly opened and a 0.5-1.0 g section of left ventricle is quickly removed and flash frozen with Wollenberger clamps cooled in liquid nitrogen for myocardial cyclic guanosine monophosphate (cGMP) analysis. The levels of cGMP in plasma are also analyzed. Then samples for histomorphometric measurements, myocardial receptor and ion channel measurements and RNA gene chip analysis are removed. Venous blood samples are obtained at the same time of the day in conscious dogs prior to each cardiac catheterization and echocardiographic measurement including days −7, 0-16, 22, 30, 38, 45, 53, 60, 68, 75, 83, and 90. Blood samples (at least 9 mL-3×3 ml) are collected in plastic tubes containing EDTA and Complete protease inhibitor (Roche Biosciences). From a stock solution of the following composition of 1 complete protease inhibitor tablet dissolved in 2 ml normal saline, each EDTA blood collection tube, contains 40 μL of complete protease inhibitor per ml whole blood. Whole blood samples are collected with EDTA and protease inhibitor. The whole blood samples collected with EDTA and protease inhibitor are immediately placed on ice and centrifuged at 3000 rpm for 10 min within 30 min of collection. The plasma is: (i) placed in cryostorage tubes; and (ii) stored upright at −70° C. until analysis to determine LANA plasma concentration. Samples of the dosing solution (2 mL) are placed in cryostorage tubes and stored upright at −70° C. Separate venous blood samples (serum) are drawn at baseline and at the end of each cardiac catherization for determination of serum electrolytes, including creatinine to estimate renal glomerular filtration rate (eGFR). Venous blood is collected at baseline and at the end of each cardiac catherization for plasma biomarkers. The dog's body weight is measured monthly just prior to each cardiac catherization.

### Hemodynamic and Angiographic Measurements

All hemodynamic measurements are made during left and right heart catheterizations in anesthetized dogs at each specified study time point. The following parameters are evaluated in all dogs: (1) aortic and LV pressures using catheter tip micromanometers (Millar Instruments); (2) peak rate of change of LV pressure during isovolumic contraction (peak+dP/dt) and relaxation (peak−dP/dt); (3) LV end diastolic pressure; (4) cardiac output; (5) stroke volume; (6) cardiac index; and (7) systemic vascular resistance.

Left ventriculograms (LV) are performed on the dogs during cardiac catheterization after completion of the hemodynamic measurements. The dogs are placed on its right side such that the left ventriculograms are recorded on digital media at 30 frames/sec during a power injection of 20 mL of contrast material (RENO M 60, Squibb Diagnostics). Correction for image magnification is made using a radiopaque grid placed at the level of the LV. LV end systolic and end diastolic volumes are calculated from angiographic silhouettes using the area length method. Premature beats and post-extrasystolic beats are excluded from the analysis. LV ejection fraction is calculated as the ratio of the difference of end diastolic (EDI) and end systolic (ESY) volumes to end diastolic volume times 100.

LV ejection fraction=[(VolumeEDI−VolumeESY)/VolumeED]×100

### Echocardiographic and Doppler Measurements

Echocardiographic and Doppler studies are performed in all dogs at all specified study time points using a VIVID 7 ultrasound system (General Electric) with a 3.5 megahertz (MHz) transducer. All echocardiographic measurements are made with the dog placed in the right lateral decubitus position and recorded on a Panasonic 6300 VHS recorder for subsequent offline analysis.

LV fractional area of shortening (FAS) and LV systolic function are measured from a short axis view at the level of the papillary muscles. LV major and minor semi-axes are measured and used for calculation of LV end-diastolic circumferential wall stress.

Wall stress is calculated as indicated below:

$$Wall Stress = Pb / h ( 1 - h / 2 b ) ( 1 - hb / 2 a2 )$$

where: P is LV end-diastolic pressure, a is LV major semi-axis, b is LV minor semi-axis, and h is LV wall thickness.

Global longitudinal strain (GLS) is measured by speckle tracking.

Mitral inflow velocity is measured by pulsed-wave Doppler echocardiography to assess LV diastolic function. The velocity waveforms is used to calculate: (i) peak mitral flow velocity in early diastole (PE); (ii) peak mitral inflow velocity during LA contraction (PA); (iii) ratio of PE to PA (PE/PA); (iv) time-velocity integral of the mitral inflow velocity waveform representing early filling (Ei), (v) time-velocity integral representing LA contraction (Ai); (vi) ratio of Ei/Ai (Ei/Ai); and (vii) deceleration time of early mitral inflow velocity (DT). Color flow Dopplers assess the presence and severity of functional mitral regurgitation (i.e., regurgitant jet). The severity of the regurgitation, when present, is quantified as the ratio of the area of the regurgitant jet to the area of the left atrium.

A 24-hour ambulatory ECG Holter monitoring, as performed at all pre-specified time points (baseline, 1, 2, 14, 30, 60, and 90 days), assesses: (1) peak; (2) average and minimum heart rate; and (3a) average number per hour of single premature beats (PVC's), (3b) couplets, (3c) triplets and (3c) episodes of ventricular tachycardia (VT) (>3 beats). An episode of non-sustained VT is defined as an episode lasting less than 30 seconds. An episode lasting more than 30 seconds is defined as “sustained VT”.

### Circulating Plasma Biomarkers

Venous blood sample(s), as obtained at baseline and at each follow-up timepoint (at baseline and following each cardiac catherization), quantify the following plasma biomarkers: (1) Troponin-I; (2) myoglobin; (3) Big-endothelin (Big-ET); (4) angiotensin-II (ANG II); (5) norepinephrine (NE); (6) N-Terminal pro-BNP (NT-pro-BNP); (7) atrial natriuretic peptide (proANP); (8) tumor necrosis factor-alpha (TNF-α); (9) interleukin-6 (IL-6); (10) C-reactive protein (CRP); (11) procollagen type 1 C-terminal propeptide (PICP); (12) CK-MB and (13) cyclic guanosine monophosphate (cGMP). Blood samples from 6 normal dogs are compared.

BIOMARKERS

| | | |
|---|---|---|
| Endothelin - 300 μL | proANP - 300 μL | High sensitivity cTNI - |
| Plasma (EDTA) | Plasma (EDTA) | 100 μL Serum |
| ANG-II - 2000 μL | TNFα - 500 μL Plasma | Renin - 500 μL Plasma |
| Plasma (EDTA) | (EDTA) | (EDTA) |
| NE - 300 μl Plasma | IL-6 - 500 μL Plasma | Big-ET -2000 μL |
| (EDTA) | (EDTA) | Plasma (EDTA) |
| NT-pro-BNP- 500 μL | CRP - 100 μL Serum | PICP- 100 μL |
| Plasma (EDTA) | | |

### Histomorphometric Measurements

From each heart, 3 transverse slices (approximately 3 mm thick) are obtained such there is one each from basal, middle and apical thirds of the LV. From each slice, transmural tissue blocks are obtained and embedded in paraffin blocks. Transmural tissue blocks, as obtained from the free wall segment of the slice, are: (i) mounted on cork using Tissue-Tek embedding medium; (ii) rapidly frozen in isopentane pre-cooled in liquid nitrogen; and (iii) stored at −70° C. until used up. The volume fraction of replacement fibrosis (VFRF), volume fraction of interstitial fibrosis (VFIF), myocyte cross-sectional area (MCSA), a measure of cardiomyocyte hypertrophy, capillary density (CD), and oxygen diffusion distance (ODD) are measured as previously described. LV tissue from 6 normal dogs is processed in an identical manner as above and the results used for comparisons.

### Myocardial Receptor and Ion Channel Measurements

From each heart, ˜1-5 g of LV anterior free wall are rapidly removed, dissected, and flash frozen at −80° C. for radioligand binding. The density and affinity of beta adrenoceptors and sarcoplasmic reticular calcium release channels are quantified by analyzing saturation isotherms from the specific binding of [³H]-dihydroalprenolol and [³H]-ryanodine to enriched sarcolemmal and sarcoplasmic reticular membranes.

### RNA Gene Chip Analysis

RNA-gene chip analysis, as used with the compositions and methods herein, involves: expression profiling, samples taken, treatment groups, and tissues. Whereby there are two samples per hound (1 for RNA, 1 for protein) and the tissues are stored in RNA later, with half kept at −70° C. for protein.

| Expression | | |
|---|---|---|
| Profiling (3) | Samples (4) | Tissues (16) |
| A. Control dog vs. | A. Control Dog Tissue | A. LV free wall |
| CHF dog | B. CHF Dog + BNP | (above CAMP sample |
| B. BNP derivative | derivative Vehicle for | region, non-scarred) |
| vehicle vs. CHF dog | 3 months treatment | B. RV free wall (above |
| C. BNP derivative | (vehicle/s.c Q5D) | CAMP sample region, |
| treatment | C. CHF Dog + BNP | non-scarred) |
| | derivative for 3 months | C. Septum (above |
| | treatment (0.1 mg/kg | CAMP sample region, |
| | s.c. Q5D) | non-scarred) |
| | D. CHF Dog + BNP | D. Coronary - LAD |
| | derivative for 3 months | (non-scarred region) |
| | treatment (0.3 mg/kg | E. Coronary - |
| | s.c. Q5D) | circumflex (non-scarred |
| | | region) |
| | | F. Aorta (descending |
| | | thoracic) |
| | | G. Adipose - epididymal |
| | | H. Adipose - mesenteric |
| | | I. Adipose - |
| | | subcutaneous |
| | | J. Adipose - |
| | | retroperitoneal |
| | | K. Adrenal medulla |
| | | L. Adrenal cortex |
| | | M. Skeletal muscle |
| | | (quadricep) |
| | | N. Skeletal muscle |
| | | (gastrocnemius) |
| | | O. Kidney (cortex)) |
| | | P. Kidney (medulla) |

### Method for Collection

Sections, which are 5 mm³, undergo dissection followed by RNALater rinsing and storage in 1-mL RNALater in labeled 1.5 mL polypropylene Eppendorf tubes. Vascular tissue (artery or vein) are collected as 1 cm lengths.

Once the data above is analyzed, it is compared to data obtained from native BNP administration and PEGylated BNP administration. It is expected that the half-life of the BNP protein is increased without the unwanted immunogenic properties which are produced by PEGylation.

### Overview of Examples 4 and 5

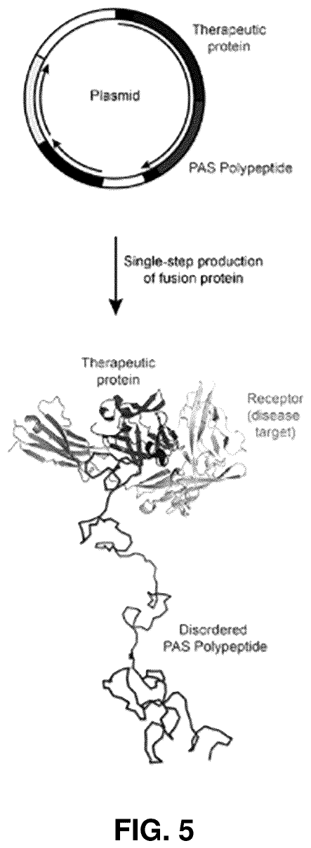

Examples 4 and 5 describe studies evaluating the biological activity of the following five PASylated BNPs: PASylated compounds 1 to 5 were prepared by recombinant means well known to those skilled in the art and illustrated in FIG. 5. Compound 1 is P-(SEQ ID No:2)₁₀-A-hBNP(1-32) (PAS attached to N-terminal amino group). Compound 2 is P-(SEQ ID No:2)₁₀-A-hBNP(3-32) (PAS attached to the alpha amino group of the N-Terminal lysine 3). Compound 3 is P-(SEQ ID No:2)₁₀-A-hBNP(6-32) (PAS attached to N-terminus of glutamine 6). Compound 4 is hBNP(1-32)-(SEQ ID No:2)₁₀-A (PAS attached to the C-terminus carboxy group). Compound 5 is hBNP(1-30)-(SEQ ID No:2)₁₀-A (PAS attached to carboxy group of the C-Terminal arginine 30).

### Example 4. NPR1 Agonist Activity of PASylated BNP

The objective of this study is to evaluate potential functional effect of test compounds on hNPR1 (the membrane-bound guanylate cyclase receptor of BNP) under agonist mode by detection of cGMP level with TR-FRET.

### Materials

| Regents | Vendor | Cat# |
|---|---|---|
| Ham's F12 | Gibco | 11765-054 |
| Fetal Bovine Serum (FBS) | GE | SH30406.05 |
| Penicillin-Streptomycin | Gibco | 15140-122 |
| Phosphate-Buffered Saline | Gibco | 14190144 |
| (PBS) | | |
| TrypLE ™Express | Gibco | 12604-013 |
| cGMP kit (Cisbio, 62GM2PEG, | Cisbio | 62GM2PEG |
| 500 tests) | | |
| Hank's Buffered Saline Solution | Gibco | 14025 |
| (HBSS) pRP[Exp]-Puro- | VectorsBuilder | 20201217HTZ13-M |
| CMV > hNPR1[NM_000906.4] | | |
| 3-Isobutyl-1-Methylxanthine | Sigma | I5879 |
| (IBMX) | | |

### Instruments and Consumables

| Item | Supplier | Cat# |
|---|---|---|
| Envision Multimode Plate Reader | PerkinElmer | 2009-0030 |
| Countess Automated Cell Counter | Invitrogen | Countess |
| STERI-CYCLE CO₂ Incubator | Thermo | 371 |
| 1300 Series Class II Biological Safety | Thermo | 1389 |
| Cabinet | | |
| Microplate Shaker | Allsheng | MX100-4A |
| 384-Well White Flat Bottom Polystyrene | Corning | 3570 |
| TC-treated Microplates | | |
| 6-well Cell Culture Plate | Corning | 3516 |
| 75 cm² Culture Flask | Corning | 430639 |
| 50 ml Centrifuge Tube | BD-Falcon | 352098 |
| 15 ml Centrifuge Tube | BD-Falcon | 352097 |
| Echo | Labcyte | 550 |

### Cell Line Information

| Cell Line | Supplier | Cat# |
|---|---|---|
| CHO-K1 | ATCC | CCL-61 |

### Experimental Procedure

### Transient Transfection

One day before the transfection, cells were harvested and the density and viability counted by using a Countess cell counter. Only cells with viability>85% were used for the following assay. Cells were seeded at a density of 9.75×10⁵ cells/dish in 6-cm dishes and cultured at 37° C., 5% (v/v) CO₂ overnight. On the following day, the growth medium was discarded and 3 ml of Opti-MEM I reduced-serum medium was added to each well.

The DNA/FuGENE 6 reagent mixture below was prepared:

| Reagent | Amount | |
|---|---|---|
| FuGENE 6 Transfection Reagent | 29.25 | μl |
| Plasmid | 9.75 | μg |

| | |
|---|---|
| Opti-MEM I Medium | up to 325 μl |

After incubating at room temperature for 15 minutes, the mixture was added to the cells and cultured at 37° C. in a humidified atmosphere with 5% (v/v) CO₂ for 6 hours. The medium was then replaced with complete culture medium (F12 medium supplemented with 10% FBS and 100 U/ml Pen-Strep) and cultured at 37° C. in a humidified atmosphere with 5% (v/v) CO₂ before use.

### Plating Cells

The growth medium was discarded 24-hour post-transfection the cells washed once with PBS. The appropriate amount of TrypLE was then added and incubated with the cells at 37° C. for 5 min. When the morphology of the cells turned round, complete growth medium was added to stop the reaction. The cells were then transferred to a sterile 15 ml centrifuge tube and centrifuged at 1200 rpm for 5 min. The supernatant was discarded and the cell pellet was resuspended in complete growth medium. The cells were counted using a Countess cell counter. Only cells with viability>85% were used for the following assay. Complete growth medium was used to dilute the cells, which were transfer to a poly-L-lysine coated 384-well plate in a density of 12000 cells/well. The cells were then cultured at 37° C. in a humidified atmosphere with 5% (v/v) CO₂ overnight.

### Agonist Assay

Reference compounds human BNP and test compounds were dissolved in ddH₂O to make 100 μM stock solutions. The growth medium was discarded and the cells were washed once with 40 μl of HBSS buffer (with Ca²⁺ and Mg²⁺). Ten μl HBSS buffer (with Ca²⁺ and Mg²⁺), supplemented with 0.5 mM IBMX, was added to each well, which were then incubated at 37° C. for 15 min. Ten nL of 3-fold serial diluted compounds were transferred from the source plate to a 384-well plate using an Echo 550. For the reference compound, the top three concentrations were prepared by transferring 300 nL, 100 nL, and 30 nL of 100 μM stock solutions. For test compounds, the top four concentrations were prepared by transferring 1000 nL, 300 nL, 100 nL, and 30 nL of 100 μM stock solutions. The plates were centrifuged at 1000 rpm for 1 min and the agitate at 600 rpm. The plates were incubated at 37° C. for 20 min. Five 5 μl/well of cGMP-d2 working solution and 5 μl/well of anti-cGMP-Eu³⁺ cryptate working solution was added to each well of the plate. The plate was then centrifuged at 1000 rpm for 1 min and the agitate at 600 rpm, then incubated at 25° C. for 1 hour.

The plate was read with an EnVison microplate reader (λex=320 nm, λem=615 nm and 665 nm), and % Activation was plotted against the concentrations of compound to build dose response curve. The curve was used to to calculate the EC₅₀ value. The results were expressed as % Activation, using the normalization equation: N=100−100×(U−C2)/(C1−C2), where U is the unknown value, C1 is the average of high controls, and C2 is the average of low controls. The lower and upper asymptotes, midpoint slope and potency (EC₅₀) are determined by fitting percentage of activation as a function of compound concentrations to a four parameter general logistic function using GraphPad Prism™ software.

### Results

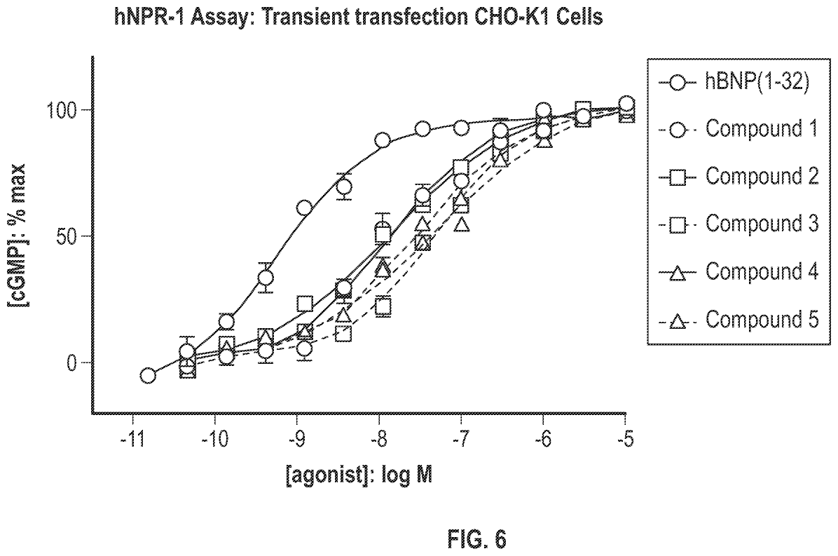

A graph of the results is provided in FIG. 6.

The results of each PASylated BNP is also shown in the following table:

| | hBNP | Compound | Compound | Compound | Compound | Compound |
|---|---|---|---|---|---|---|
| compound | (1-32) | 1 | 2 | 3 | 4 | 5 |
| log EC50 | −9.23 | −7.88 | −7.84 | −7.31 | −7.56 | −7.28 |
| s.e.m. log | 0.09 | 0.07 | 0.07 | 0.05 | 0.05 | 0.08 |
| EC50 | | | | | | |
| log dose | | −1.35 | −1.39 | −1.92 | −1.67 | −1.95 |
| ratio from | | | | | | |
| hBNP(1-32) | | | | | | |
| s.e.m log | | 0.11 | 0.11 | 0.10 | 0.10 | 0.12 |
| dose ratio | | | | | | |
| potency fold | | 22 | 25 | 83 | 47 | 89 |
| loss from | | | | | | |
| hBNP(1-32) | | | | | | |

### Discussion

This example established that the PASylated BNP has agonist activity at the human NPR1.

### Example 5. Evaluation of the Potency of hBNP(1-32) and Compound 1 in Carbachol Pre-Contracted Isolated Guinea-Pig Tracheal Rings

### Methods

All animals received standard housing and husbandry with water and food ad libitum. Male albino guinea pigs (Dunkin-Hartley; 400-700 g; Envigo, Horst, NL) were sacrificed by inhalation of CO₂ followed by exsanguination. The trachea was gently dissected from the surrounding connective tissue, cut into eight intact rings of equal length containing between two and three cartilage rings each and placed in a 5 mL tissue organ bath containing Krebs-Ringer PSS (2.5 mM CaCl₂)) and kept at 37° C., constantly bubbled with carbogen (5% CO₂ in O₂) to maintain the pH at 7.4.

The tension (mN) was measured continuously via an isometric transducer following the slow adjustment of the resting force to 30 mN. As a control of guinea pig tracheal reactivity, histamine (0.1 nM to 0.3 mM) was cumulatively added. Before the pharmacological investigation, indomethacin (3 μM) was added to prevent release and possible interference of prostaglandins. Following a resting period of 30 minutes, the segments were contracted with 3 nM of the muscarinic agonist carbachol to give a stable pre-contraction of around 50-75% of maximal contraction. When the contractile response had attained a plateau, a relaxation concentration-response curve was obtained in each ring by cumulative addition of hBNP(1-32) or Compound 1 at 0.5 log unit dose intervals (0.1 nM-1 μM) for each agonist, randomized by allocation across organs baths and experimental runs. Responses were expressed as percentage decrease in tension relative to the initial contractile response to carbachol. These experiments were ended with a concentration combination of papaverine (0.1 mM) and sodium nitroprusside (0.1 mM) as to determine maximal relaxation.

### Calculations and Statistics

All data are presented as mean #S.E.M. Data were fitted by non-linear regression to a 3-parameter general logistic to calculate potency (pEC₅₀), midpoint slope and upper asymptote values. Statistical analysis was performed using unpaired t-test for comparisons between two groups using GraphPad Prism 8.2.1 software (GraphPad Software Inc., San Diego, CA, U.S.A.). A p-value of <0.05 was deemed significant.

### Drugs and Suppliers

Stock solutions and dilutions were prepared according to manufacturers and suppliers' instructions. NaHCO₃, CaCl₂) and KCl were obtained from VWR (West Chester, PA, U.S.A.).

Histamine dihydrochloride, carbachol, papaverine, sodium nitroprusside, Krebs-Henseleit PSS Buffer, indomethacin and human brain natriuretic peptide, hBNP(1-32), were purchased from Sigma-Aldrich (St. Louis, MO, U.S.A.). All stock solutions were stored at −20° C. Dilutions of drugs were freshly made from stocks prior to each experiment in reducing concentrations of its solvent (Krebs-Ringer PSS).

### Results

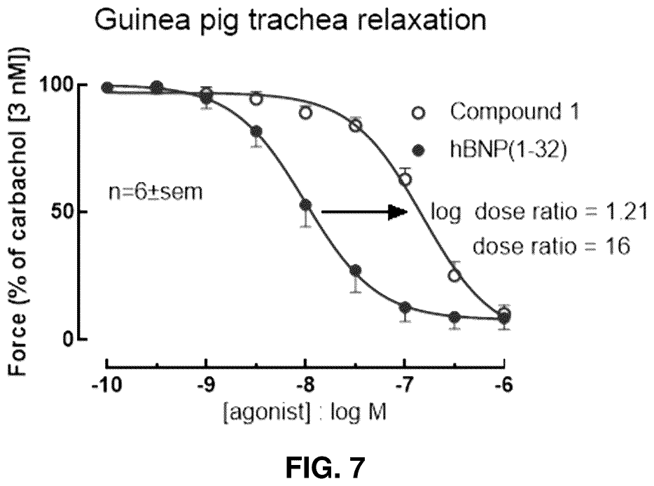

In the guinea pig tracheal segments, 3 nM carbachol induced a stable pre-contraction with a maximal loss of contraction that was 4±6% during the experimental time course (FIG. 7). Cumulative administration of hBNP(1-32) and Compound 1 on top of the pre-contraction induced parallel concentration-dependent relaxations (slope values: 1.3±0.1, and 1.3±0.3, respectively) with similar maximal effect (Emax: 91±4% and 90±3%, respectively). The potency for hBNP(1-32) (pEC₅₀: 8.00±0.10) was 16-fold (16.3±1.3) higher than for Compound 1 (pEC₅₀: 6.79±0.05; p<0.0001).

### Discussion

This Example further establishes that PASylated BNP retains BNP biological activity in physiological tissue.

### Example 6. A Single Dose Pharmacokinetic/Pharmacodynamic Study of Compound 1 Following Subcutaneous and Intravenous Administration in Beagle Dogs

The objective of this study was to determine the pharmacokinetic/pharmacodynamic (PKPD) profile of Compound 1 for 6 days following a single dose of Compound 1 after subcutaneous and intravenous administration in Beagle dogs.

### Study Design

Compound 1 was dissolved in phosphate buffered saline at a concentration of 0.4 mg/ml for intravenous dosing and 1.8 mg/kg for subcutaneous dosing. Group 1 received a subcutaneous bolus 0.5 mL/kg dose of phosphate buffered saline. Group 2 received a subcutaneous bolus dose of 0.9 mg/kg Compound 1 in a volume of 0.5 mL/kg. Group 3 received an intravenous bolus dose of 0.2 mg/kg Compound 1 in a volume of 0.5 mL/kg. There were three male animals in each group.

Animals were randomized on a weight stratified basis so that a comparable distribution of body weights among groups was achieved after randomization (within ±20% of the mean weight value; overall individual weight range 8-13 kg). The absolute dose volumes for individual animals were calculated based on the animals' most recently recorded body weights.

All the animals were surgically implanted with telemetric transmitters for continuous recording of arterial blood pressure and heart rate for the first 24-hour period following dosing using an EMKA telemetry system, IOX2 software and a BP-2010E Non-Invasive Blood Pressure (NIBP) Monitor. After 24 hours post-dosing, blood pressure and heart rate were measured by cuff manometry at 24-hour intervals (day 3-6).

Blood samples were drawn at the following timepoints in all three treatment groups: pre-dose (−10 min), 10 min, 30 min, 1 h, 2 h, 3 h, 4 h, 6 h, 8 h, 12 h, 16 h, 24 h, 48 h, 72 h, 96 h, 120 h and 144 h (post-dose). The 0.5 mL samples were collected by venepuncture at each time point from each animal and placed in potassium (K₂) EDTA treated tubes which were stoppered and gently inverted several times to ensure anticoagulation. These were stored on ice for a maximum of 60 minutes before being centrifuged at approximately 2,000 g for 10 minutes at 4° C. to allow withdrawal of the plasma. The plasma was split into two aliquots (equal volumes) and transferred to cryogenic vials. It was then stored at −75° C. One set of plasma was used for determination of the concentration of Compound 1 using a sandwich ELISA setup with the high-affinity monoclonal αPAS antibody Avi-PA(S) 1.1 and the αhBNP antibody clone 50E1 (specificity for the C-terminus of hBNP32) ensuring high sensitivity and selectivity. The second set of plasma was used for the determination of plasma cGMP levels using an Abcam ELISA kit (Cyclic GMP Complete ELISA Kit (ab133052)|Abcam).

### Results

### Pharmacokinetics

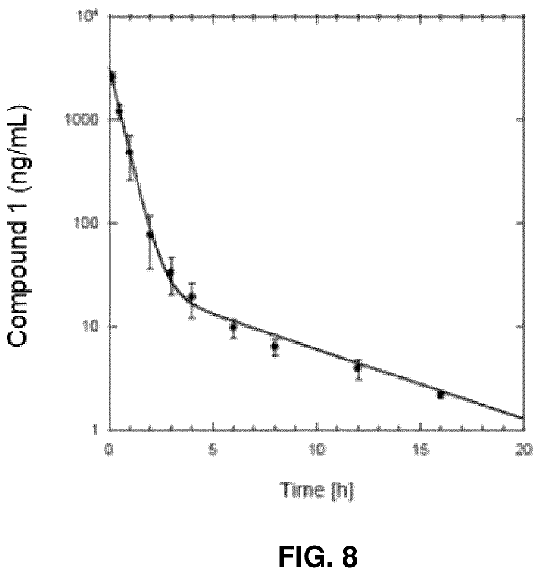

The data from the ELISA assay of Group 3, 0.2 mg/kg intravenous bolus Compound 1, plasma samples could be fitted to a typical Bateman function. The results of the analysis are shown in FIG. 8 and Table 1.

TABLE 1
Pharmacokinetic parameter values from a two-compartment model fit
of canine plasma concentrations of Compound 1 over time following
intravenous bolus dosing (0.2 mg/kg).

| Parameter | |
|---|---|
| Cmax (ng/ml) | 3246.6 ± 356 |
| AUC_{0-∞} (h ng/ml) | 1814.5 ± 128 |
| t_{1/2}α (min) | 21.3 ± 1.1 |
| t_{1/2}β (min) | 255 ± 23 |
| CL (ml/h/kg) | 330.7 ± 23 |

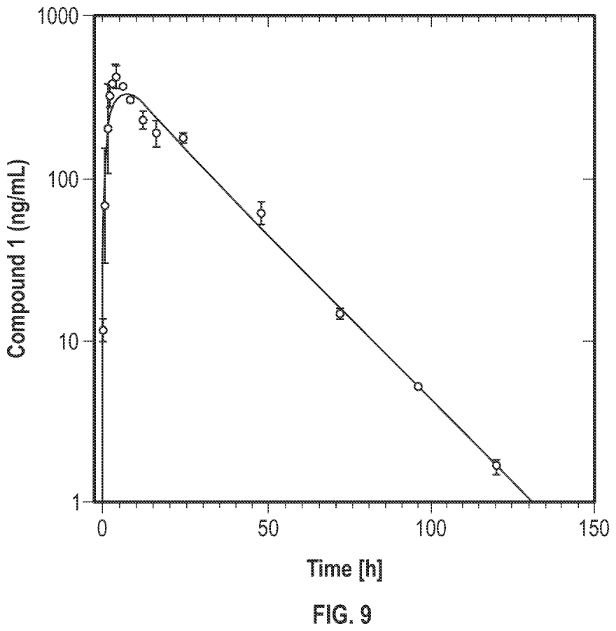

The data from the ELISA assay of Group 2 (0.9 mg/kg subcutaneous bolus Compound 1 plasma samples showed a typical Bateman function biphasic pharmacokinetic profile and was fitted to a two-compartment model. The results of the analysis are shown in FIG. 9 and Table 2.

TABLE 2
Pharmacokinetic parameter values from a two-compartment model fit
of canine plasma concentrations of Compound 1 over time following
subcutaneous bolus dosing (0.9 mg/kg).

| Parameter | |
|---|---|
| kₐ (1/h) | 0.19 |
| Cmax (ng/ml) | 303.8 |
| AUC_{0-∞} (h ng/ml) | 8818.5 |
| t_{1/2}α (h) | 3.7 |
| t_{1/2}β (h) | 14.8 |
| CL (ml/h/kg) | 102.1 |

The terminal half-life following subcutaneous dosing is 14.8 hours which is 27-fold longer than that reported for the parent peptide, hBNP(1-32) (33 minutes, reference: FDA NDA #20-920 Pharmr P1 page 28, Drug Approval Package: Natrecor (Nesiritide) NDA #20-920 (fda.gov))

### Pharmacodynamics

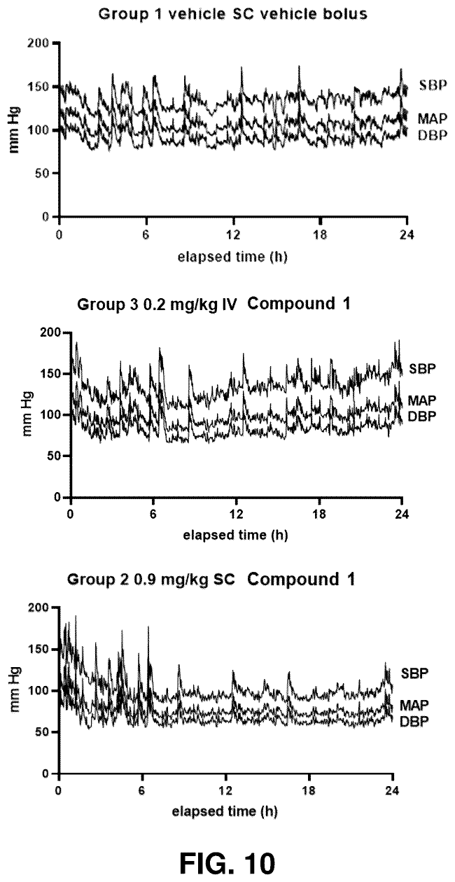

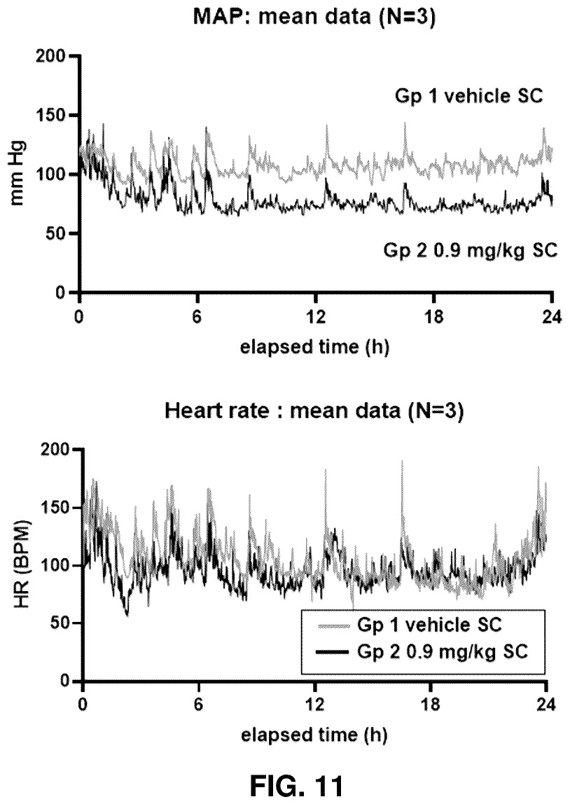

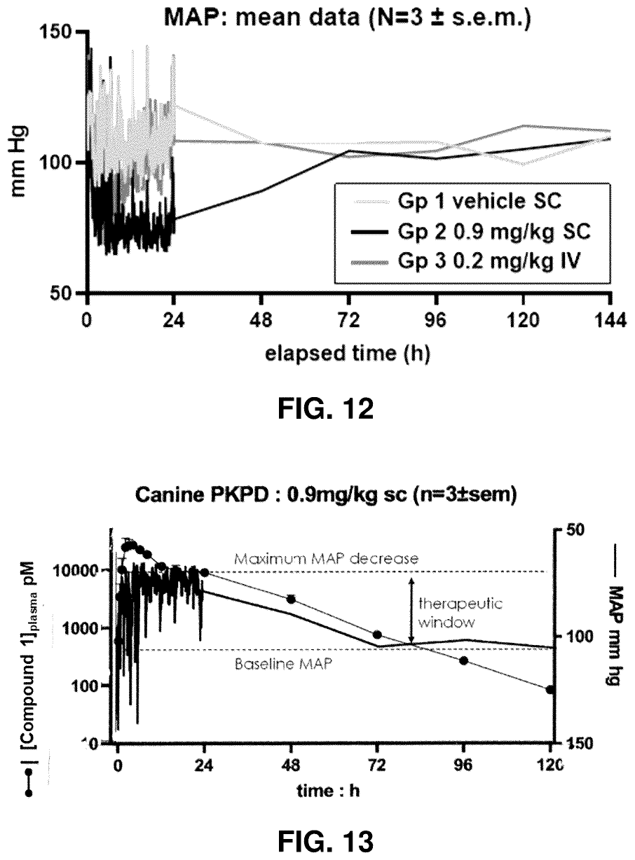

0.9 mg/kg subcutaneous bolus Compound 1 produced a sustained significant (P<0.05) decrease in systolic blood pressure (SBP), diastolic blood pressure (DBP) and calculated mean arterial pressure (MAP=DBP+[0.33+(HR×0.0012)]×[SBP]) without a significant effect on the heart rate (FIGS. 10 and 11). The mean decrease in MAP between 6 and 12 hours post dosing was 32.4±6.1 mm Hg. Over the same, 6-to-12-hour, period the heart rate was decreased by 7±13 beats per minute (not significant). The MAP returned to baseline and vehicle control levels after 72 hours (FIG. 12).

The intravenous bolus dose of 0.2 mg/kg Compound 1 also produced a significant transient decrease in blood pressure which returned to baseline at 24 hours post dose (FIG. 12).

### Pharmacokinetics/Pharmacodynamics

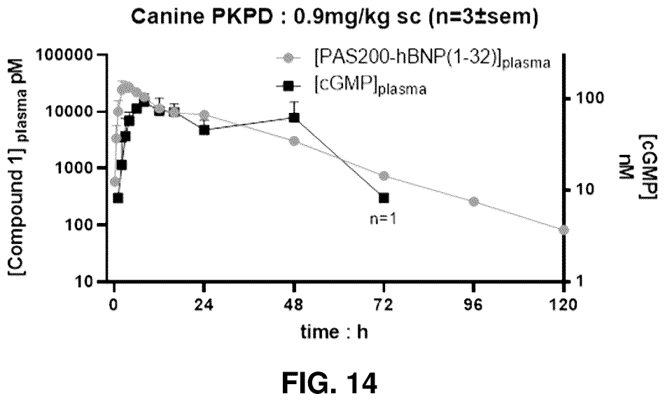

Both the effect on MAP (FIG. 13) and the concentrations of the biomarker, plasma cGMP, (FIG. 14) mirrored the plasma concentrations of Compound 1 following administration of the 0.9 mg/kg subcutaneous bolus of Compound 1. The data in FIG. 13 also shows that the 0.9 mg/kg subcutaneous bolus dose produced plasma concentrations over the duration of the study that defined the therapeutic window for the compound from sub-threshold pharmacological effect levels to supramaximal effect levels.

### Example 7. Characteristics of PAS200, PAS400, PAS600 and PAS800

PAS200 (“PAS200-hBNP(1-32)”, “Compound 1” described above) (SEQ ID NO:32), PAS400 (“PAS400-hBNP(1-32)”) (SEQ ID NO:33), PAS600 (“PAS600-hBNP(1-32)”) (SEQ ID NO:34), and PAS800 (“PAS800-hBNP(1-32)”) (SEQ ID NO:35), having about 200, 400, 600, and 800 amino acid long repeats of SEQ ID NO:2, respectively, covalently bound to the N-terminus of hBNP(1-32)(SEQ ID NO:1), were prepared by standard methods similar to those described in Examples 1 and 2 above. Purity was greater than 98% in all cases, as determined by SDS-PAGE. Expected mass (20150.6 Da, 36669.1 Da, 53187.5 Da, and 59705.9 Da respectively) was confirmed by ESI-MS.

### Example 8. In-Vitro NPR-1 cGMP Assay: Transient Transfection of HEK293T/17 Cells

### Materials and Methods

HEK293-T17 cells (ATCC CRL-11268) were cultured in a t75 cm² flask at 37° C., 5% (v/v) CO₂. On reaching 70% confluence (day 2), the growth medium was discarded and 5 ml of Opti-MEM I reduced-serum medium was added to the cells. The flask was then returned to the incubator. DNA/PEI reagent mixture was prepared at a weight ratio of 1:3. The DNA/PEI reagent mixture was incubated at room temperature for 20 min before being added to the cells. The cells were further cultured at 37° C. in a humidified atmosphere with 5% (v/v) CO₂ for 16 hours (day 3). At this point, the medium was supplemented with complete culture medium (DMEM with 10% FBS and 100 U/ml Pen-Strep). The transfected cells were then further cultured at 37° C. in a humidified atmosphere with 5% (v/v) CO₂ for a further 24 h before use (on day 4).

The growth medium was then discarded 48 hours post-transfection and the cells were washed once with 10 mL PBS. 2 mL of TrypLE was then added to the cells and incubated at 37° C. for 2 min, the point at which the cells round up. Complete growth medium (8 mL) was added to stop the reaction and the cells transferred to a sterile 25 mL centrifuge tube (Universal). The cells were counted using a Countess cell counter. An appropriate amount of cells (˜5 million) were then centrifuged at 350 g for 3 min. The cell pellet was resuspended in complete assay buffer (HBSS buffer, 0.1% BSA, 5 mM HEPES supplemented with 0.5 mM IBMX) and then transferred to a 384-well Optiplate plate to a final density of 18,000 cells/well in a total volume of 4.5 μL. The plates were then centrifuged at 100 g for 30 seconds and incubated at 37° C. for 15 min.

Reference compound human brain natriuretic peptide 1-32 (hBNP(1-32)) was initially dissolved in water at a concentration of 1 mM, and test compounds were dissolved in PBS to make 100 μM stock solutions. The hBNP(1-32) was further diluted in PBS to a concentration of 100 μM, with all further dilutions of peptides made in assay buffer. Specifically, peptides were serially diluted in assay buffer in 3-fold steps in low-bind polypropylene tubes and transferred (0.5 μL) to the assay plate. The plates were then agitated at 600 rpm for 30 seconds. The assay plates were then incubated at 37° C. for 20 min.

cGMP standard was prepared according to the kit manufacturer's instructions, in assay buffer with 5 μL of each dilution added to the assay plate. 2.5 μL/well of cGMP-d2 working solution and 2.5 μL/well of Anti-cGMP-Eu3+ cryptate working solution was then added to each well of the plate. The plate was centrifuged at 1000 rpm for 30 seconds and then agitated (Eppendorf MixMate) at 600 rpm before being incubated at 25° C. for 1 hour.

The plate and the samples were then transferred to the Pherastar FSX with HTRF ratios (FI 665/620*10,000) monitored using 10 flashes for up to 7 cycles. Associated data was exported to EXCEL and analyzed in Prism. Concentration response data was plotted in Prism as % response to BNP1-32 (10 μM) and pEC₅₀ and Emax value were calculated. The upper asymptotes, midpoint slope and potency (EC₅₀) were determined by fitting percentage of activation as a function of compound concentrations to a four parameter general logistic function using GraphPad Prism™ software.

### Results

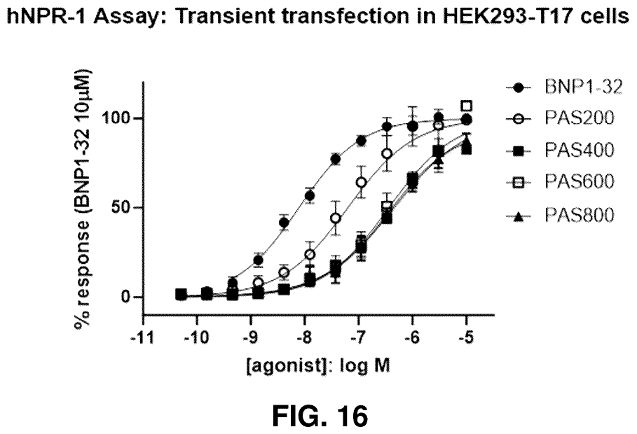

PAS200, PAS400, PAS600, PAS800 and hBNP(1-32) were dissolved in PBS and diluted in cGMP assay buffer (57 pM-10 μM) for the detection of cGMP in HEK293-T17 hNPR cell samples. FIG. 16 represents the mean normalized cGMP responses measured in transiently transfected HEK293/T17 cells with absolute cGMP levels calculated from the cGMP Std curve.

Each assay point represents the results of 5-7 separate determinations performed in singlet.

Tables 3 and 4 shows the calculated potency and intrinsic activity of the five compounds as pEC₅₀, Hill slope, Maximum % control; Table 4 shows the potency of the four analogs relative to human hBNP.

TABLE 3
Potency and intrinsic activity of human BNP-32 and PAS analogues.
Data are presented as mean ± SEM.

| | PEC₅₀ (n) | Hill slope | Maximum % control |
|---|---|---|---|
| hBNP1-32 | 8.07 ± 0.09 (6) | 0.79 ± 0.06 | 100.1 ± 3.5 |
| PAS200 | 7.21 ± 0.16 (7) | 0.81 ± 0.09 | 104.9 ± 14.0 |
| PAS400 | 6.49 ± 0.21 (5) | 0.83 ± 0.11 | 97.3 ± 10.4 |
| PAS600 | 6.57 ± 0.23 (5) | 0.85 ± 0.10 | 97.4 ± 13.5 |
| PAS800 | 6.53 ± 0.16 (5) | 0.82 ± 0.09 | 89.1 ± 5.2 |

TABLE 4
Potency of PAS analogues relative to human BNP-32.
Data are presented as mean ± SEM.

| | hBNP | | | | |
|---|---|---|---|---|---|
| Compound | (1-32) | PAS200 | PAS400 | PAS600 | PAS800 |
| Log EC50 | 8.07 | 7.21 | 6.49 | 6.57 | 6.53 |
| s.e.m. log | 0.09 | 0.16 | 0.21 | 0.23 | 0.16 |
| EC50 | | | | | |
| Log dose | | 0.86 | 1.58 | 1.50 | 1.54 |
| ratio from | | | | | |
| hBNP(1-32) | | | | | |
| s.e.m. log | | 0.18 | 0.23 | 0.25 | 0.20 |
| dose ratio | | | | | |
| Potency fold | | 7.3 | 38.2 | 31.8 | 34.3 |
| loss from | | | | | |
| hBNP(1-32) | | | | | |

### Discussion

The PAS200 analog was 7.3-fold less potent than the native peptide, hBNP(1-32), at the NPR1. Doubling the PAS size resulted in a further 5.2-fold reduction in potency so that the PAS400 analog was 38.2-fold less potent than the native peptide. Surprisingly, increasing the PAS sizes a further 1.5-fold (PAS600) and 2-fold again (PAS800) did not produce any further loss of potency.

### Example 9. A Single Dose Pharmacokinetic/Pharmacodynamic Study of PAS200, PAS400, PAS600, and PAS800 Following Subcutaneous (SC) and Intravenous (IV) Bolus Administration in Rats

The objective of this study was to determine the pharmacokinetic profile of PAS200, PAS400, PAS600 and PAS800 for 7 days following a single dose of each after subcutaneous and intravenous bolus administration in normotensive rats.

### Materials and Methods

### Study Design

The study was carried in 8 male Han Wistar rats supplied by Envigo, UK divided into two groups of 4 for IV and SC dosing respectively. The animals were dosed in a cross-over design so that animals received a single dose of vehicle control or a dose of hBNP(1-32) analogue on Day 1 of each week. There was a washout interval of 7 days between successive doses.

### Dosing

The test items for subcutaneous SC dosing were formulated by adding sterile phosphate-buffered saline (PBS) to vials containing pre-weighed hBNP(1-32) analogs to achieve a concentration of 83.5 nmol/mL. Aliquots of these solutions were diluted by removing 0.4 mL and adding to a vial containing 1.4 mL of PBS. The diluted solutions, at a concentration of 18.6 nmol/mL, were used for IV dosing.

The SC dose for all hBNP(1-32) analogs was 167 nmol/kg, at a volume of 2 mL/kg, equivalent to 3.376 mg/kg PAS200-hBNP(1-32), 6.142 mg/kg PAS400-hBNP(1-32), 8.902 mg/kg PAS600-hBNP(1-32) and 11.662 mg/kg PAS800-hBNP(1-32).

The IV dose for all hBNP(1-32) analogs was 18.6 nmol/kg in in a volume of 1 mL/kg, equivalent to 0.375 mg/kg PAS200-hBNP(1-32), 0.682 mg/kg PAS400-hBNP(1-32), 0.989 mg/kg PAS600-hBNP(1-32) and 1.296 mg/kg PAS800-hBNP(1-32).

### Blood Sampling

Blood samples were taken on dosing days (Day 1) at approximately 0.5 h, 2 h, 4 h, 8 h and 12 h post-dose for the SC dosed animals and at 10 min, 1 h, 4 h, 8 h and 12 h post-dose for the IV dosed animals. For both SC and IV doses, samples were also taken at approximately 24 h (Day 2), 48 h (Day 3), 72 h (Day 4), 96 h (Day 5), 120 h (Day 6) and 144 h (Day 7). Samples were collected by tail prick using capillary micro-sampling and placed in potassium (K₂) EDTA treated tubes which were stoppered and gently inverted several times to ensure anticoagulation. These were stored on ice for a maximum of 60 minutes before being centrifuged at approximately 2,000 g for 10 minutes at 4° C. to allow withdrawal of the plasma which was transferred to cryogenic vials. It was then stored at −75° C. The plasma concentrations of the compounds were determined using a sandwich ELISA setup with the high-affinity monoclonal anti-PAS antibody Avi-PA(S) 1.1 and the anti-humanBNP antibody clone 50E1 (specificity for the C-terminus of hBNP32) ensuring high sensitivity and selectivity.

### Results

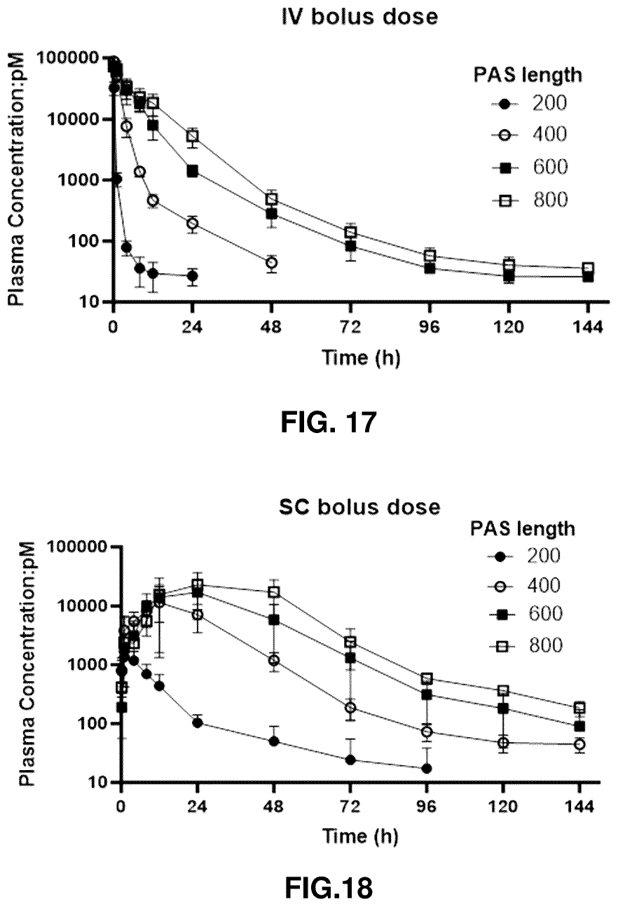

The plasma concentrations from the ELISA assay of plasma samples following IV and SC dosing are shown over time in FIG. 17 and FIG. 18. Both sets of data were fitted to a non-compartmental model using Phoenix WinNonlin software (Certara) and the results of the analysis are shown in Table 5.

TABLE 5
Pharmacokinetic parameter values (t1/2beta, AUC, CL/F) from a non-compartmental
model fit of rat plasma values over time of PAS200, PAS400, PAS600, PAS800
following intravenous and subcutaneous bolus dosing (mean +/− S.D., N = 4) together
with the empirical Tmax and Cmax values from the subcutaneous data in FIG. 18.

| Route | t1/2beta: h | AUC: h*pM | CL/F: mg/(h*pM)/kg | Tmax: | Cmax: pM |
|---|---|---|---|---|---|
| CPD ID | (S.D) | (S.D) | (S.D) | h | (SEM) |
| SC PAS200 | 1.51 (0.32) | 18608 (3527) | 2.078E−05 (3.29E−6) | 1 | 1364 (322) |
| SC PAS400 | 12.00 (2.00) | 310861 (149287) | 2.359E−05 (8.35E−06) | 12 | 11395 (4891) |
| SC PAS600 | 27.59 (6.55) | 655625 (369304) | 5.864E−05 (8.325E−05) | 24 | 17002 (5537) |
| SC PAS800 | 30.15 (12.51) | 1070383 (583155) | 1.448E−05 (6.87E−06) | 24 | 22856 (7115) |
| IV PAS200 | 7.11 (3.80) | 14828 (4235) | 2.459E−04 (6.46E−05) | | |
| IV PAS400 | 11.24 (2.90) | 461393 (35605) | 3.848E−06 (7.37E−07) | | |
| IV PAS600 | 38.90 (18.73) | 436745 (79274) | 2.351E−06 (4.81E−07) | | |
| IV PAS800 | 27.40 (3.34) | 632838 (159754) | 2.184E−06 (5.57E−07) | | |

### Discussion

The terminal half-life estimates for the PAS400, PAS600 and PAS800 analogs were similar whether estimated from the IV or SC dosing data. The estimate from the PAS200 data was numerically higher from the IV data (7.11 hours) than from the SC data (1.51 hours). However, the terminal phase of the plasma concentration-time curve (FIG. 17) was poorly defined following IV dosing and therefore unreliable due to the relatively rapid decline in plasma levels to below the limit of detection of the ELISA assay.

The native human peptide, hBNP(1-32), has a half-life of 6.4 minutes in rat (Semenov et al., 2011). The half-life estimates from the SC dosing data are 14-fold (PAS200), 112-fold (PAS400), 258-fold (PAS600) and 281-fold (PAS800) longer than for hBNP(1-32). The effect of increasing PAS size on half-life extension appears to reach a plateau with only a 1.1-fold increase between PAS600 and PAS800 compared to a 14-fold increase between hBNP(1-32) and PAS200, 8-fold between PAS200 and PAS400 and 2.3-fold between PAS400 and PAS600.

The surprisingly large increases in half-life of the PAS200, PAS400, PAS600 and PAS800 compounds vs. hBNP(1-32), when administered SC in rats, were unexpected, since the increases in rat SC half-life with analogous leptin, Fab, Interferon, and human growth hormone (hGH) PAS compounds was much less. Table 6.

TABLE 6
Half-life increase of PASylated compound over unPASylated compound,
in multiples of unPASylated compound (fold-increase)

| | PAS-200 | PAS-400 | PAS-600 | PAS-800 |
|---|---|---|---|---|
| BNP^{a} | 14 | 112 | 258 | 281 |
| Leptin^{b} | 7.7 | 16 | 45.6 | — |
| Fab^{c} | 3.9 | 10.7 | 21.0 | — |
| Interferon^{c} | 4.9 | 14.6 | 29.4 | — |
| hGH^{c} | — | — | 94 | — |

ªThis study;
^{b}Morath et al., 2015;
^{c}Schlapschy et al., 2015

The significant increase in Tmax with increasing PAS size progressive increase in Tmax is indicative of changes beyond simple half-life extension suggesting PAS-size dependent changes in rates of absorption from the SC site of administration and effects on distribution.

The behavior of other PASylated proteins and proteins shows the effect of increasing PAS size on both agonist receptor binding/potency and pharmacokinetic profile (overall changes in half-life, AUC, Clearance, Cmax and Tmax following extravascular dosing are highly specific to the compound being PASylated (Table 6).

Sequences
SEQ ID NO: 36
SPKMVQGSGCFGRKMDRISSSSGLGCKVLRRH
SEQ ID NO. 2
ASPAAPAPASPAAPAPSAPA
SEQ ID NO: 3
AAPASPAPAAPSAPAPAAPS
SEQ ID NO: 4
APSSPSPSAPSSPSPASPSS
SEQ ID NO: 5
SAPSSPSPSAPSSPSPASPS
SEQ ID NO: 6
SSPSAPSPSSPASPSPSSPA
SEQ ID NO: 7
AASPAAPSAPPAAASPAAPSAPPA
SEQ ID NO: 8
ASAAAPAAASAAASAPSAAA
SEQ ID NO: 9
APAAPAPAPAAPAPAPA
SEQ ID NO: 10
AAPAPAPAAPAPAPAAP
SEQ ID NO: 11
APPPAPPPAP
SEQ ID NO: 12
PAPPPAPPPA
SEQ ID NO: 13
AAPAAPAPPAAAPAAPAPPA
SEQ ID NO: 14
AAAAPAAAAAAAPAAA
SEQ ID NO: 37
CPKMVQGSGCFGRKMDRISSSSGLGCKVLRRH
SEQ ID NO: 38
SPCMVQGSGCFGRKMDRISSSSGLGCKVLRRH
SEQ ID NO: 39
SPKCVQGSGCFGRKMDRISSSSGLGCKVLRRH
SEQ ID NO: 40
SPKMCQGSGCFGRKMDRISSSSGLGCKVLRRH
SEQ ID NO: 41
SPKMVCGSGCFGRKMDRISSSSGLGCKVLRRH
SEQ ID NO: 42
SPKMVQGCGCFGRKMDRISSSSGLGCKVLRRH
(Synth. gene fragment SapI-BNP32)
SEQ ID NO: 21
AGGTAACATATGCCTGCCAGAAGAGCTCCTCAGCGCTCTTCTGCCAGTCCGAAAATG
GTTCAAGGTAGCGGTTGTTTTGGTCGTAAAATGGATCGTATTAGCAGCAGCAGCGGT
CTGGGTTGTAAAGTTCTGCGTCGTCATTAATAAGCTTGGGTTG
(Synth. gene fragment SapI-BNP3-32)
SEQ ID NO: 22
AGGTAAcAtATGCCAGCCAGAAGAGCTCCTCAGCGCTCTTCTGCCAAAATGGTTCAA
GGTAGCGGTTGTTTTGGTCGTAAAATGGATCGTATTAGCAGCAGCAGCGGTCTGGGT
TGTAAAGTTCTGCGTCGTCATTAATAAGCTTGGGTTG
(Synth. gene fragment SapI-BNP6-32)
SEQ ID NO: 23
ATTCGTTCAGGTAAcAtATGCCAGCCAGAAGAGCTCCTCAGCGCTCTTCTGCCCAAGG
TAGCGGTTGTTTTGGTCGTAAAATGGATCGTATTAGCAGCAGCAGCGGTCTGGGTTG
TAAAGTTCTGCGTCGTCATTAATAAGCTTGGGTTG
(Synth. gene fragment BNP32)
SEQ ID NO: 24
AGGTAAcAtATGAGTCCGAAAATGGTTCAAGGTAGCGGTTGTTTTGGTCGTAAAATG
GATCGTATTAGCAGCAGCAGCGGTCTGGGTTGTAAAGTTCTGCGTCGTCATGCCAGA
AGAGCTCCTCAGCGCTCTTCTGCCTAATAAGCTTGGGTTG
(Synth. gene fragment BNP1-30)
SEQ ID NO: 25
AGGTAAcAtATGAGTCCGAAAATGGTTCAAGGTAGCGGTTGTTTTGGTCGTAAAATG
GATCGTATTAGCAGCAGCAGCGGTCTGGGTTGTAAAGTTCTGCGTGCCAGAAGAGCT
CCTCAGCGCTCTTCTGCCTAATAAGCTTGGGTTG
(PAS1.2(200))
SEQ ID NO: 26
GCCAGCCCTGCCGCACCTGCGCCCGCATCACCTGCGGCACCTGCACCTTCCGCCCCG
GCTGCATCTCCTGCCGCACCCGCGCCTGCCAGCCCAGCTGCACCTGCCCCAAGTGCG
CCAGCAGCATCCCCTGCCGCGCCTGCCCCCGCTAGTCCAGCGGCCCCAGCTCCATCT
GCACCAGCTGCTAGCCCTGCTGCACCAGCTCCTGCTTCTCCCGCAGCCCCAGCGCCT
TCTGCTCCCGCAGCCTCACCTGCGGCCCCGGCACCAGCATCTCCAGCGGCACCAGCA
CCTTCGGCCCCTGCTGCTAGCCCAGCAGCACCTGCGCCAGCCTCACCAGCTGCTCCC
GCTCCTAGTGCCCCGGCGGCCTCGCCTGCTGCTCCTGCACCAGCTTCGCCAGCGGCA
CCGGCTCCTTCGGCGCCGGCTGCTTCACCAGCAGCACCTGCTCCAGCGTCCCCAGCG
GCCCCTGCTCCAAGTGCTCCGGCTGCATCGCCTGCCGCTCCTGCTCCTGCATCCCCA
GCTGCTCCAGCACCAAGCGCACCTGCCGCCTCACCAGCGGCGCCAGCACCCGCCAG
CCCAGCAGCGCCTGCTCCATCCGCACCGGCGGCC
(pD451-SR-PAS200-BNP32)
SEQ ID NO: 27
CCCGTAGAAAAGATCAAAGGATCTTCTTGAGATCCTTTTTTTCTGCGCGTAATCTGCT
GCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACT
GTTCTTCTAGTGTAGCCGTAGTTAGCCCACCACTTCAAGAACTCTGTAGCACCGCCT
ACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGAAC
TGAGATACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACT
TGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTTCTTT
CCTGCGTTATCCCCTGATTCTGTGGATAACCGTATTACCGCCTTTGAGTGAGCTGATA
CCGCTCGCCGCAGCCGAACGACCGAGCGCAGCGAGTCAGTGAGCGAGGAAGCGGA
AGGCGAGAGTAGGGAACTGCCAGGCATCAAACTAAGCAGAAGGCCCCTGACGGAT
GGCCTTTTTGCGTTTCTACAAACTCTTTCTGTGTTGTAAAACGACGGCCAGTCTTAAG
CTCGGGCCCCCTGGGCGGTTCTGATAACGAGTAATCGTTAATCCGCAAATAACGTAA
AAACCCGCTTCGGCGGGTTTTTTTATGGGGGGAGTTTAGGGAAAGAGCATTTGTCAG
AATATTTAAGGGCGCCTGTCACTTTGCTTGATATATGAGAATTATTTAACCTTATAAA
TGAGAAAAAAGCAACGCACTTTAAATAAGATACGTTGCTTTTTCGATTGATGAACAC
CTATAATTAAACTATTCATCTATTATTTATGATTTTTTGTATATACAATATTTCTAGTT
TGTTAAAGAGAATTAAGAAAATAAATCTCGAAAATAATAAAGGGAAAATCAGTTTT
TGATATCAAAATTATACATGTCAACGATAATACAAAATATAATACAAACTATAAGAT
GTTATCAGTATTTATTATGCATTTAGAATAAATTTTGTGTCGCCCTTCCGCGAAATTA
ATACGACTCACTATAGGGGAATTGTGAGCGGATAACAATTCCCCTCTAGAAATAATT
TTGTTTAACTTTTTGAGACCTTAAGGAGGTAAAACATATGCCTgccagccctgccgcacctgcgc
ccgcatcacctgcggcacctgcaccttccgccccggctgcatctcctgccgcacccgcgcctgccagcccagctgcacctgccccaagtg
cgccagcagcatcccctgccgcgcctgcccccgctagtccagcggccccagctccatctgcaccagctgctagccctgctgcaccagctc
ctgcttctcccgcagccccagcgccttctgctcccgcagcctcacctgcggccccggcaccagcatctccagcggcaccagcaccttcgg
cccctgctgctagcccagcagcacctgcgccagcctcaccagctgctcccgctcctagtgccccggcggcctcgcctgctgctcctgcac
cagcttcgccagcggcaccggctccttcggcgccggctgcttcaccagcagcacctgctccagcgtccccagcggcccctgctccaagtg
ctccggctgcatcgcctgccgctcctgctcctgcatccccagctgctccagcaccaagcgcacctgccgcctcaccagcggcgccagcac
ccgccagcccagcagcgcctgctccatccgcaccggcgGCCAGTCCGAAAATGGTTCAAGGTAGCGGTTG
TTTTGGTCGTAAAATGGATCGTATTAGCAGCAGCAGCGGTCTGGGTTGTAAAGTTCT
GCGTCGTCATTAATAAGCTTGGTTGAGGTCTCACCCCCTAGCATAACCCCTTGGGGC
CTCTAAACGGGTCTTGAGGGGTTTTTTGCCCCTGAGACGCGTCAATCGAGTTCGTAC
CTAAGGGCGACACCCCCTAATTAGCCCGGGCGAAAGGCCCAGTCTTTCGACTGAGC
CTTTCGTTTTATTTGATGCCTGGCAGTTCCCTACTCTCGCATGGGGAGTCCCCACACT
ACCATCGGCGCTACGGCGTTTCACTTCTGAGTTCGGCATGGGGTCAGGTGGGACCAC
CGCGCTACTGCCGCCAGGCAAACAAGGGGTGTTATGAGCCATATTCAGGTATAAAT
GGGCTCGCGATAATGTTCAGAATTGGTTAATTGGTTGTAACACTGACCCCTATTTGTT
TATTTTTCTAAATACATTCAAATATGTATCCGCTCATGAGACAATAACCCTGATAAA
TGCTTCAATAATATTGAAAAAGGAAGAATATGAGCCATATTCAACGGGAAACGTCG
AGGCCGCGATTAAATTCCAACATGGATGCTGATTTATATGGGTATAAATGGGCTCGC
GATAATGTCGGGCAATCAGGTGCGACAATCTATCGCTTGTATGGGAAGCCCGATGC
GCCAGAGTTGTTTCTGAAACATGGCAAAGGTAGCGTTGCCAATGATGTTACAGATGA
GATGGTCAGACTAAACTGGCTGACGGAATTTATGCCACTTCCGACCATCAAGCATTT
TATCCGTACTCCTGATGATGCATGGTTACTCACCACTGCGATCCCCGGAAAAACAGC
GTTCCAGGTATTAGAAGAATATCCTGATTCAGGTGAAAATATTGTTGATGCGCTGGC
AGTGTTCCTGCGCCGGTTGCACTCGATTCCTGTTTGTAATTGTCCTTTTAACAGCGAT
CGCGTATTTCGCCTCGCTCAGGCGCAATCACGAATGAATAACGGTTTGGTTGATGCG
AGTGATTTTGATGACGAGCGTAATGGCTGGCCTGTTGAACAAGTCTGGAAAGAAAT
GCATAAACTTTTGCCATTCTCACCGGATTCAGTCGTCACTCATGGTGATTTCTCACTT
GATAACCTTATTTTTGACGAGGGGAAATTAATAGGTTGTATTGATGTTGGACGAGTC
GGAATCGCAGACCGATACCAGGATCTTGCCATCCTATGGAACTGCCTCGGTGAGTTT
TCTCCTTCATTACAGAAACGGCTTTTTCAAAAATATGGTATTGATAATCCTGATATGA
ATAAATTGCAGTTTCATTTGATGCTCGATGAGTTTTTCTAAGCGGCGCGCCATCGAA
TGGCGCAAAACCTTTCGCGGTATGGCATGATAGCGCCCGGAAGAGAGTCAATTCAG
GGTGGTGAATATGAAACCAGTAACGTTATACGATGTCGCAGAGTATGCCGGTGTCTC
TTATCAGACCGTTTCCCGCGTGGTGAACCAGGCCAGCCACGTTTCTGCGAAAACGCG
GGAAAAAGTGGAAGCGGCGATGGCGGAGCTGAATTACATTCCCAACCGCGTGGCAC
AACAACTGGCGGGCAAACAGTCGTTGCTGATTGGCGTTGCCACCTCCAGTCTGGCCC
TGCACGCGCCGTCGCAAATTGTCGCGGCGATTAAATCTCGCGCCGATCAACTGGGTG
CCAGCGTGGTGGTGTCGATGGTAGAACGAAGCGGCGTCGAAGCCTGTAAAGCGGCG
GTGCACAATCTTCTCGCGCAACGCGTCAGTGGGCTGATCATTAACTATCCGCTGGAT
GACCAGGATGCCATTGCTGTGGAAGCTGCCTGCACTAATGTTCCGGCGTTATTTCTT
GATGTCTCTGACCAGACACCCATCAACAGTATTATTTTCTCCCATGAGGACGGTACG
CGACTGGGCGTGGAGCATCTGGTCGCATTGGGTCACCAGCAAATCGCGCTGTTAGCG
GGCCCATTAAGTTCTGTCTCGGCGCGTCTGCGTCTGGCTGGCTGGCATAAATATCTC
ACTCGCAATCAAATTCAGCCGATAGCGGAACGGGAAGGCGACTGGAGTGCCATGTC
CGGTTTTCAACAAACCATGCAAATGCTGAATGAGGGCATCGTTCCCACTGCGATGCT
GGTTGCCAACGATCAGATGGCGCTGGGCGCAATGCGCGCCATTACCGAGTCCGGGC
TGCGCGTTGGTGCGGATATCTCGGTAGTGGGATACGACGATACCGAAGATAGCTCAT
GTTATATCCCGCCGTTAACCACCATCAAACAGGATTTTCGCCTGCTGGGGCAAACCA
GCGTGGACCGCTTGCTGCAACTCTCTCAGGGCCAGGCGGTGAAGGGCAATCAGCTG
TTGCCAGTCTCACTGGTGAAAAGAAAAACCACCCTGGCGCCCAATACGCAAACCGC
CTCTCCCCGCGCGTTGGCCGATTCATTAATGCAGCTGGCACGACAGGTTTCCCGACT
GGAAAGCGGGCAGTGACTCATGACCAAAATCCCTTAACGTGAGTTACGCGCGCGTC
GTTCCACTGAGCGTCAGAC
(pD451-SR-PAS200-BNP3-32)
SEQ ID NO: 28
CCCGTAGAAAAGATCAAAGGATCTTCTTGAGATCCTTTTTTTCTGCGCGTAATCTGCT
GCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACT
GTTCTTCTAGTGTAGCCGTAGTTAGCCCACCACTTCAAGAACTCTGTAGCACCGCCT
ACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGAAC
TGAGATACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACT
TGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTTCTTT
CCTGCGTTATCCCCTGATTCTGTGGATAACCGTATTACCGCCTTTGAGTGAGCTGATA
CCGCTCGCCGCAGCCGAACGACCGAGCGCAGCGAGTCAGTGAGCGAGGAAGCGGA
AGGCGAGAGTAGGGAACTGCCAGGCATCAAACTAAGCAGAAGGCCCCTGACGGAT
GGCCTTTTTGCGTTTCTACAAACTCTTTCTGTGTTGTAAAACGACGGCCAGTCTTAAG
CTCGGGCCCCCTGGGCGGTTCTGATAACGAGTAATCGTTAATCCGCAAATAACGTAA
AAACCCGCTTCGGCGGGTTTTTTTATGGGGGGAGTTTAGGGAAAGAGCATTTGTCAG
AATATTTAAGGGCGCCTGTCACTTTGCTTGATATATGAGAATTATTTAACCTTATAAA
TGAGAAAAAAGCAACGCACTTTAAATAAGATACGTTGCTTTTTCGATTGATGAACAC
CTATAATTAAACTATTCATCTATTATTTATGATTTTTTGTATATACAATATTTCTAGTT
TGTTAAAGAGAATTAAGAAAATAAATCTCGAAAATAATAAAGGGAAAATCAGTTTT
TGATATCAAAATTATACATGTCAACGATAATACAAAATATAATACAAACTATAAGAT
GTTATCAGTATTTATTATGCATTTAGAATAAATTTTGTGTCGCCCTTCCGCGAAATTA
ATACGACTCACTATAGGGGAATTGTGAGCGGATAACAATTCCCCTCTAGAAATAATT
TTGTTTAACTTTTTGAGACCTTAAGGAGGTAAAACATATGCCAgccagccctgccgcacctgcgc
ccgcatcacctgcggcacctgcaccttccgccccggctgcatctcctgccgcacccgcgcctgccagcccagctgcacctgccccaagtg
cgccagcagcatcccctgccgcgcctgcccccgctagtccagcggccccagctccatctgcaccagctgctagccctgctgcaccagctc
ctgcttctcccgcagccccagcgccttctgctcccgcagcctcacctgcggccccggcaccagcatctccagcggcaccagcaccttcgg
cccctgctgctagcccagcagcacctgcgccagcctcaccagctgctcccgctcctagtgccccggcggcctcgcctgctgctcctgcac
cagcttcgccagcggcaccggctccttcggcgccggctgcttcaccagcagcacctgctccagcgtccccagcggcccctgctccaagtg
ctccggctgcatcgcctgccgctcctgctcctgcatccccagctgctccagcaccaagcgcacctgccgcctcaccagcggcgccagcac
ccgccagcccagcagcgcctgctccatccgcaccggcgGCCAAAATGGTTCAAGGTAGCGGTTGTTTTGG
TCGTAAAATGGATCGTATTAGCAGCAGCAGCGGTCTGGGTTGTAAAGTTCTGCGTCG
TCATTAATAAGCTTGGTTGAGGTCTCACCCCCTAGCATAACCCCTTGGGGCCTCTAA
ACGGGTCTTGAGGGGTTTTTTGCCCCTGAGACGCGTCAATCGAGTTCGTACCTAAGG
GCGACACCCCCTAATTAGCCCGGGCGAAAGGCCCAGTCTTTCGACTGAGCCTTTCGT
TTTATTTGATGCCTGGCAGTTCCCTACTCTCGCATGGGGAGTCCCCACACTACCATCG
GCGCTACGGCGTTTCACTTCTGAGTTCGGCATGGGGTCAGGTGGGACCACCGCGCTA
CTGCCGCCAGGCAAACAAGGGGTGTTATGAGCCATATTCAGGTATAAATGGGCTCG
CGATAATGTTCAGAATTGGTTAATTGGTTGTAACACTGACCCCTATTTGTTTATTTTT
CTAAATACATTCAAATATGTATCCGCTCATGAGACAATAACCCTGATAAATGCTTCA
ATAATATTGAAAAAGGAAGAATATGAGCCATATTCAACGGGAAACGTCGAGGCCGC
GATTAAATTCCAACATGGATGCTGATTTATATGGGTATAAATGGGCTCGCGATAATG
TCGGGCAATCAGGTGCGACAATCTATCGCTTGTATGGGAAGCCCGATGCGCCAGAG
TTGTTTCTGAAACATGGCAAAGGTAGCGTTGCCAATGATGTTACAGATGAGATGGTC
AGACTAAACTGGCTGACGGAATTTATGCCACTTCCGACCATCAAGCATTTTATCCGT
ACTCCTGATGATGCATGGTTACTCACCACTGCGATCCCCGGAAAAACAGCGTTCCAG
GTATTAGAAGAATATCCTGATTCAGGTGAAAATATTGTTGATGCGCTGGCAGTGTTC
CTGCGCCGGTTGCACTCGATTCCTGTTTGTAATTGTCCTTTTAACAGCGATCGCGTAT
TTCGCCTCGCTCAGGCGCAATCACGAATGAATAACGGTTTGGTTGATGCGAGTGATT
TTGATGACGAGCGTAATGGCTGGCCTGTTGAACAAGTCTGGAAAGAAATGCATAAA
CTTTTGCCATTCTCACCGGATTCAGTCGTCACTCATGGTGATTTCTCACTTGATAACC
TTATTTTTGACGAGGGGAAATTAATAGGTTGTATTGATGTTGGACGAGTCGGAATCG
CAGACCGATACCAGGATCTTGCCATCCTATGGAACTGCCTCGGTGAGTTTTCTCCTTC
ATTACAGAAACGGCTTTTTCAAAAATATGGTATTGATAATCCTGATATGAATAAATT
GCAGTTTCATTTGATGCTCGATGAGTTTTTCTAAGCGGCGCGCCATCGAATGGCGCA
AAACCTTTCGCGGTATGGCATGATAGCGCCCGGAAGAGAGTCAATTCAGGGTGGTG
AATATGAAACCAGTAACGTTATACGATGTCGCAGAGTATGCCGGTGTCTCTTATCAG
ACCGTTTCCCGCGTGGTGAACCAGGCCAGCCACGTTTCTGCGAAAACGCGGGAAAA
AGTGGAAGCGGCGATGGCGGAGCTGAATTACATTCCCAACCGCGTGGCACAACAAC
TGGCGGGCAAACAGTCGTTGCTGATTGGCGTTGCCACCTCCAGTCTGGCCCTGCACG
CGCCGTCGCAAATTGTCGCGGCGATTAAATCTCGCGCCGATCAACTGGGTGCCAGCG
TGGTGGTGTCGATGGTAGAACGAAGCGGCGTCGAAGCCTGTAAAGCGGCGGTGCAC
AATCTTCTCGCGCAACGCGTCAGTGGGCTGATCATTAACTATCCGCTGGATGACCAG
GATGCCATTGCTGTGGAAGCTGCCTGCACTAATGTTCCGGCGTTATTTCTTGATGTCT
CTGACCAGACACCCATCAACAGTATTATTTTCTCCCATGAGGACGGTACGCGACTGG
GCGTGGAGCATCTGGTCGCATTGGGTCACCAGCAAATCGCGCTGTTAGCGGGCCCAT
TAAGTTCTGTCTCGGCGCGTCTGCGTCTGGCTGGCTGGCATAAATATCTCACTCGCA
ATCAAATTCAGCCGATAGCGGAACGGGAAGGCGACTGGAGTGCCATGTCCGGTTTT
CAACAAACCATGCAAATGCTGAATGAGGGCATCGTTCCCACTGCGATGCTGGTTGCC
AACGATCAGATGGCGCTGGGCGCAATGCGCGCCATTACCGAGTCCGGGCTGCGCGT
TGGTGCGGATATCTCGGTAGTGGGATACGACGATACCGAAGATAGCTCATGTTATAT
CCCGCCGTTAACCACCATCAAACAGGATTTTCGCCTGCTGGGGCAAACCAGCGTGG
ACCGCTTGCTGCAACTCTCTCAGGGCCAGGCGGTGAAGGGCAATCAGCTGTTGCCA
GTCTCACTGGTGAAAAGAAAAACCACCCTGGCGCCCAATACGCAAACCGCCTCTCC
CCGCGCGTTGGCCGATTCATTAATGCAGCTGGCACGACAGGTTTCCCGACTGGAAAG
CGGGCAGTGACTCATGACCAAAATCCCTTAACGTGAGTTACGCGCGCGTCGTTCCAC
TGAGCGTCAGAC
(pD451-SR-PAS200-BNP6-32)
SEQ ID NO: 29
CCCGTAGAAAAGATCAAAGGATCTTCTTGAGATCCTTTTTTTCTGCGCGTAATCTGCT
GCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACT
GTTCTTCTAGTGTAGCCGTAGTTAGCCCACCACTTCAAGAACTCTGTAGCACCGCCT
ACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGAAC
TGAGATACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACT
TGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTTCTTT
CCTGCGTTATCCCCTGATTCTGTGGATAACCGTATTACCGCCTTTGAGTGAGCTGATA
CCGCTCGCCGCAGCCGAACGACCGAGCGCAGCGAGTCAGTGAGCGAGGAAGCGGA
AGGCGAGAGTAGGGAACTGCCAGGCATCAAACTAAGCAGAAGGCCCCTGACGGAT
GGCCTTTTTGCGTTTCTACAAACTCTTTCTGTGTTGTAAAACGACGGCCAGTCTTAAG
CTCGGGCCCCCTGGGCGGTTCTGATAACGAGTAATCGTTAATCCGCAAATAACGTAA
AAACCCGCTTCGGCGGGTTTTTTTATGGGGGGAGTTTAGGGAAAGAGCATTTGTCAG
AATATTTAAGGGCGCCTGTCACTTTGCTTGATATATGAGAATTATTTAACCTTATAAA
TGAGAAAAAAGCAACGCACTTTAAATAAGATACGTTGCTTTTTCGATTGATGAACAC
CTATAATTAAACTATTCATCTATTATTTATGATTTTTTGTATATACAATATTTCTAGTT
TGTTAAAGAGAATTAAGAAAATAAATCTCGAAAATAATAAAGGGAAAATCAGTTTT
TGATATCAAAATTATACATGTCAACGATAATACAAAATATAATACAAACTATAAGAT
GTTATCAGTATTTATTATGCATTTAGAATAAATTTTGTGTCGCCCTTCCGCGAAATTA
ATACGACTCACTATAGGGGAATTGTGAGCGGATAACAATTCCCCTCTAGAAATAATT
TTGTTTAACTTTTTGAGACCTTAAGGAGGTAAAACATATGCCAgccagccctgccgcacctgcgc
ccgcatcacctgcggcacctgcaccttccgccccggctgcatctcctgccgcacccgcgcctgccagcccagctgcacctgccccaagtg
cgccagcagcatcccctgccgcgcctgcccccgctagtccagcggccccagctccatctgcaccagctgctagccctgctgcaccagctc
ctgcttctcccgcagccccagcgccttctgctcccgcagcctcacctgcggccccggcaccagcatctccagcggcaccagcaccttcgg
cccctgctgctagcccagcagcacctgcgccagcctcaccagctgctcccgctcctagtgccccggcggcctcgcctgctgctcctgcac
cagcttcgccagcggcaccggctccttcggcgccggctgcttcaccagcagcacctgctccagcgtccccagcggcccctgctccaagtg
ctccggctgcatcgcctgccgctcctgctcctgcatccccagctgctccagcaccaagcgcacctgccgcctcaccagcggcgccagcac
ccgccagcccagcagcgcctgctccatccgcaccggcgGCCCAAGGTAGCGGTTGTTTTGGTCGTAAAAT
GGATCGTATTAGCAGCAGCAGCGGTCTGGGTTGTAAAGTTCTGCGTCGTCATTAATA
AGCTTGGTTGAGGTCTCACCCCCTAGCATAACCCCTTGGGGCCTCTAAACGGGTCTT
GAGGGGTTTTTTGCCCCTGAGACGCGTCAATCGAGTTCGTACCTAAGGGCGACACCC
CCTAATTAGCCCGGGCGAAAGGCCCAGTCTTTCGACTGAGCCTTTCGTTTTATTTGAT
GCCTGGCAGTTCCCTACTCTCGCATGGGGAGTCCCCACACTACCATCGGCGCTACGG
CGTTTCACTTCTGAGTTCGGCATGGGGTCAGGTGGGACCACCGCGCTACTGCCGCCA
GGCAAACAAGGGGTGTTATGAGCCATATTCAGGTATAAATGGGCTCGCGATAATGT
TCAGAATTGGTTAATTGGTTGTAACACTGACCCCTATTTGTTTATTTTTCTAAATACA
TTCAAATATGTATCCGCTCATGAGACAATAACCCTGATAAATGCTTCAATAATATTG
AAAAAGGAAGAATATGAGCCATATTCAACGGGAAACGTCGAGGCCGCGATTAAATT
CCAACATGGATGCTGATTTATATGGGTATAAATGGGCTCGCGATAATGTCGGGCAAT
CAGGTGCGACAATCTATCGCTTGTATGGGAAGCCCGATGCGCCAGAGTTGTTTCTGA
AACATGGCAAAGGTAGCGTTGCCAATGATGTTACAGATGAGATGGTCAGACTAAAC
TGGCTGACGGAATTTATGCCACTTCCGACCATCAAGCATTTTATCCGTACTCCTGATG
ATGCATGGTTACTCACCACTGCGATCCCCGGAAAAACAGCGTTCCAGGTATTAGAAG
AATATCCTGATTCAGGTGAAAATATTGTTGATGCGCTGGCAGTGTTCCTGCGCCGGT
TGCACTCGATTCCTGTTTGTAATTGTCCTTTTAACAGCGATCGCGTATTTCGCCTCGC
TCAGGCGCAATCACGAATGAATAACGGTTTGGTTGATGCGAGTGATTTTGATGACGA
GCGTAATGGCTGGCCTGTTGAACAAGTCTGGAAAGAAATGCATAAACTTTTGCCATT
CTCACCGGATTCAGTCGTCACTCATGGTGATTTCTCACTTGATAACCTTATTTTTGAC
GAGGGGAAATTAATAGGTTGTATTGATGTTGGACGAGTCGGAATCGCAGACCGATA
CCAGGATCTTGCCATCCTATGGAACTGCCTCGGTGAGTTTTCTCCTTCATTACAGAAA
CGGCTTTTTCAAAAATATGGTATTGATAATCCTGATATGAATAAATTGCAGTTTCATT
TGATGCTCGATGAGTTTTTCTAAGCGGCGCGCCATCGAATGGCGCAAAACCTTTCGC
GGTATGGCATGATAGCGCCCGGAAGAGAGTCAATTCAGGGTGGTGAATATGAAACC
AGTAACGTTATACGATGTCGCAGAGTATGCCGGTGTCTCTTATCAGACCGTTTCCCG
CGTGGTGAACCAGGCCAGCCACGTTTCTGCGAAAACGCGGGAAAAAGTGGAAGCGG
CGATGGCGGAGCTGAATTACATTCCCAACCGCGTGGCACAACAACTGGCGGGCAAA
CAGTCGTTGCTGATTGGCGTTGCCACCTCCAGTCTGGCCCTGCACGCGCCGTCGCAA
ATTGTCGCGGCGATTAAATCTCGCGCCGATCAACTGGGTGCCAGCGTGGTGGTGTCG
ATGGTAGAACGAAGCGGCGTCGAAGCCTGTAAAGCGGCGGTGCACAATCTTCTCGC
GCAACGCGTCAGTGGGCTGATCATTAACTATCCGCTGGATGACCAGGATGCCATTGC
TGTGGAAGCTGCCTGCACTAATGTTCCGGCGTTATTTCTTGATGTCTCTGACCAGACA
CCCATCAACAGTATTATTTTCTCCCATGAGGACGGTACGCGACTGGGCGTGGAGCAT
CTGGTCGCATTGGGTCACCAGCAAATCGCGCTGTTAGCGGGCCCATTAAGTTCTGTC
TCGGCGCGTCTGCGTCTGGCTGGCTGGCATAAATATCTCACTCGCAATCAAATTCAG
CCGATAGCGGAACGGGAAGGCGACTGGAGTGCCATGTCCGGTTTTCAACAAACCAT
GCAAATGCTGAATGAGGGCATCGTTCCCACTGCGATGCTGGTTGCCAACGATCAGAT
GGCGCTGGGCGCAATGCGCGCCATTACCGAGTCCGGGCTGCGCGTTGGTGCGGATA
TCTCGGTAGTGGGATACGACGATACCGAAGATAGCTCATGTTATATCCCGCCGTTAA
CCACCATCAAACAGGATTTTCGCCTGCTGGGGCAAACCAGCGTGGACCGCTTGCTGC
AACTCTCTCAGGGCCAGGCGGTGAAGGGCAATCAGCTGTTGCCAGTCTCACTGGTGA
AAAGAAAAACCACCCTGGCGCCCAATACGCAAACCGCCTCTCCCCGCGCGTTGGCC
GATTCATTAATGCAGCTGGCACGACAGGTTTCCCGACTGGAAAGCGGGCAGTGACT
CATGACCAAAATCCCTTAACGTGAGTTACGCGCGCGTCGTTCCACTGAGCGTCAGAC
(pD451-SR-BNP32-PAS200)
SEQ ID NO: 30
CCCGTAGAAAAGATCAAAGGATCTTCTTGAGATCCTTTTTTTCTGCGCGTAATCTGCT
GCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACT
GTTCTTCTAGTGTAGCCGTAGTTAGCCCACCACTTCAAGAACTCTGTAGCACCGCCT
ACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGAAC
TGAGATACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACT
TGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTTCTTT
CCTGCGTTATCCCCTGATTCTGTGGATAACCGTATTACCGCCTTTGAGTGAGCTGATA
CCGCTCGCCGCAGCCGAACGACCGAGCGCAGCGAGTCAGTGAGCGAGGAAGCGGA
AGGCGAGAGTAGGGAACTGCCAGGCATCAAACTAAGCAGAAGGCCCCTGACGGAT
GGCCTTTTTGCGTTTCTACAAACTCTTTCTGTGTTGTAAAACGACGGCCAGTCTTAAG
CTCGGGCCCCCTGGGCGGTTCTGATAACGAGTAATCGTTAATCCGCAAATAACGTAA
AAACCCGCTTCGGCGGGTTTTTTTATGGGGGGAGTTTAGGGAAAGAGCATTTGTCAG
AATATTTAAGGGCGCCTGTCACTTTGCTTGATATATGAGAATTATTTAACCTTATAAA
TGAGAAAAAAGCAACGCACTTTAAATAAGATACGTTGCTTTTTCGATTGATGAACAC
CTATAATTAAACTATTCATCTATTATTTATGATTTTTTGTATATACAATATTTCTAGTT
TGTTAAAGAGAATTAAGAAAATAAATCTCGAAAATAATAAAGGGAAAATCAGTTTT
TGATATCAAAATTATACATGTCAACGATAATACAAAATATAATACAAACTATAAGAT
GTTATCAGTATTTATTATGCATTTAGAATAAATTTTGTGTCGCCCTTCCGCGAAATTA
ATACGACTCACTATAGGGGAATTGTGAGCGGATAACAATTCCCCTCTAGAAATAATT
TTGTTTAACTTTTTGAGACCTTAAGGAGGTAAAACATATGAGTCCGAAAATGGTTCA
AGGTAGCGGTTGTTTTGGTCGTAAAATGGATCGTATTAGCAGCAGCAGCGGTCTGGG
TTGTAAAGTTCTGCGTCGTCATgccagccctgccgcacctgcgcccgcatcacctgcggcacctgcaccttccgccc
cggctgcatctcctgccgcacccgcgcctgccagcccagctgcacctgccccaagtgcgccagcagcatcccctgccgcgcctgccccc
gctagtccagcggccccagctccatctgcaccagctgctagccctgctgcaccagctcctgcttctcccgcagccccagcgccttctgct
cccgcagcctcacctgcggccccggcaccagcatctccagcggcaccagcaccttcggcccctgctgctagcccagcagcacctgcgcca
gcctcaccagctgctcccgctcctagtgccccggcggcctcgcctgctgctcctgcaccagcttcgccagcggcaccggctccttcggcg
ccggctgcttcaccagcagcacctgctccagcgtccccagcggcccctgctccaagtgctccggctgcatcgcctgccgctcctgctcctg
catccccagctgctccagcaccaagcgcacctgccgcctcaccagcggcgccagcacccgccagcccagcagcgcctgctccatccgc
accggcgGCCTAATAAGCTTGGTTGAGGTCTCACCCCCTAGCATAACCCCTTGGGGCCT
CTAAACGGGTCTTGAGGGGTTTTTTGCCCCTGAGACGCGTCAATCGAGTTCGTACCT
AAGGGCGACACCCCCTAATTAGCCCGGGCGAAAGGCCCAGTCTTTCGACTGAGCCT
TTCGTTTTATTTGATGCCTGGCAGTTCCCTACTCTCGCATGGGGAGTCCCCACACTAC
CATCGGCGCTACGGCGTTTCACTTCTGAGTTCGGCATGGGGTCAGGTGGGACCACCG
CGCTACTGCCGCCAGGCAAACAAGGGGTGTTATGAGCCATATTCAGGTATAAATGG
GCTCGCGATAATGTTCAGAATTGGTTAATTGGTTGTAACACTGACCCCTATTTGTTTA
TTTTTCTAAATACATTCAAATATGTATCCGCTCATGAGACAATAACCCTGATAAATG
CTTCAATAATATTGAAAAAGGAAGAATATGAGCCATATTCAACGGGAAACGTCGAG
GCCGCGATTAAATTCCAACATGGATGCTGATTTATATGGGTATAAATGGGCTCGCGA
TAATGTCGGGCAATCAGGTGCGACAATCTATCGCTTGTATGGGAAGCCCGATGCGCC
AGAGTTGTTTCTGAAACATGGCAAAGGTAGCGTTGCCAATGATGTTACAGATGAGAT
GGTCAGACTAAACTGGCTGACGGAATTTATGCCACTTCCGACCATCAAGCATTTTAT
CCGTACTCCTGATGATGCATGGTTACTCACCACTGCGATCCCCGGAAAAACAGCGTT
CCAGGTATTAGAAGAATATCCTGATTCAGGTGAAAATATTGTTGATGCGCTGGCAGT
GTTCCTGCGCCGGTTGCACTCGATTCCTGTTTGTAATTGTCCTTTTAACAGCGATCGC
GTATTTCGCCTCGCTCAGGCGCAATCACGAATGAATAACGGTTTGGTTGATGCGAGT
GATTTTGATGACGAGCGTAATGGCTGGCCTGTTGAACAAGTCTGGAAAGAAATGCA
TAAACTTTTGCCATTCTCACCGGATTCAGTCGTCACTCATGGTGATTTCTCACTTGAT
AACCTTATTTTTGACGAGGGGAAATTAATAGGTTGTATTGATGTTGGACGAGTCGGA
ATCGCAGACCGATACCAGGATCTTGCCATCCTATGGAACTGCCTCGGTGAGTTTTCT
CCTTCATTACAGAAACGGCTTTTTCAAAAATATGGTATTGATAATCCTGATATGAAT
AAATTGCAGTTTCATTTGATGCTCGATGAGTTTTTCTAAGCGGCGCGCCATCGAATG
GCGCAAAACCTTTCGCGGTATGGCATGATAGCGCCCGGAAGAGAGTCAATTCAGGG
TGGTGAATATGAAACCAGTAACGTTATACGATGTCGCAGAGTATGCCGGTGTCTCTT
ATCAGACCGTTTCCCGCGTGGTGAACCAGGCCAGCCACGTTTCTGCGAAAACGCGG
GAAAAAGTGGAAGCGGCGATGGCGGAGCTGAATTACATTCCCAACCGCGTGGCACA
ACAACTGGCGGGCAAACAGTCGTTGCTGATTGGCGTTGCCACCTCCAGTCTGGCCCT
GCACGCGCCGTCGCAAATTGTCGCGGCGATTAAATCTCGCGCCGATCAACTGGGTGC
CAGCGTGGTGGTGTCGATGGTAGAACGAAGCGGCGTCGAAGCCTGTAAAGCGGCGG
TGCACAATCTTCTCGCGCAACGCGTCAGTGGGCTGATCATTAACTATCCGCTGGATG
ACCAGGATGCCATTGCTGTGGAAGCTGCCTGCACTAATGTTCCGGCGTTATTTCTTG
ATGTCTCTGACCAGACACCCATCAACAGTATTATTTTCTCCCATGAGGACGGTACGC
GACTGGGCGTGGAGCATCTGGTCGCATTGGGTCACCAGCAAATCGCGCTGTTAGCG
GGCCCATTAAGTTCTGTCTCGGCGCGTCTGCGTCTGGCTGGCTGGCATAAATATCTC
ACTCGCAATCAAATTCAGCCGATAGCGGAACGGGAAGGCGACTGGAGTGCCATGTC
CGGTTTTCAACAAACCATGCAAATGCTGAATGAGGGCATCGTTCCCACTGCGATGCT
GGTTGCCAACGATCAGATGGCGCTGGGCGCAATGCGCGCCATTACCGAGTCCGGGC
TGCGCGTTGGTGCGGATATCTCGGTAGTGGGATACGACGATACCGAAGATAGCTCAT
GTTATATCCCGCCGTTAACCACCATCAAACAGGATTTTCGCCTGCTGGGGCAAACCA
GCGTGGACCGCTTGCTGCAACTCTCTCAGGGCCAGGCGGTGAAGGGCAATCAGCTG
TTGCCAGTCTCACTGGTGAAAAGAAAAACCACCCTGGCGCCCAATACGCAAACCGC
CTCTCCCCGCGCGTTGGCCGATTCATTAATGCAGCTGGCACGACAGGTTTCCCGACT
GGAAAGCGGGCAGTGACTCATGACCAAAATCCCTTAACGTGAGTTACGCGCGCGTC
GTTCCACTGAGCGTCAGAC
(pD451-SR-BNP1-30-PAS200)
SEQ ID NO: 31
CCCGTAGAAAAGATCAAAGGATCTTCTTGAGATCCTTTTTTTCTGCGCGTAATCTGCT
GCTTGCAAACAAAAAAACCACCGCTACCAGCGGTGGTTTGTTTGCCGGATCAAGAG
CTACCAACTCTTTTTCCGAAGGTAACTGGCTTCAGCAGAGCGCAGATACCAAATACT
GTTCTTCTAGTGTAGCCGTAGTTAGCCCACCACTTCAAGAACTCTGTAGCACCGCCT
ACATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGAAC
TGAGATACCTACAGCGTGAGCTATGAGAAAGCGCCACGCTTCCCGAAGGGAGAAAG
GCGGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTGACT
TGAGCGTCGATTTTTGTGATGCTCGTCAGGGGGGCGGAGCCTATGGAAAAACGCCA
GCAACGCGGCCTTTTTACGGTTCCTGGCCTTTTGCTGGCCTTTTGCTCACATGTTCTTT
CCTGCGTTATCCCCTGATTCTGTGGATAACCGTATTACCGCCTTTGAGTGAGCTGATA
CCGCTCGCCGCAGCCGAACGACCGAGCGCAGCGAGTCAGTGAGCGAGGAAGCGGA
AGGCGAGAGTAGGGAACTGCCAGGCATCAAACTAAGCAGAAGGCCCCTGACGGAT
GGCCTTTTTGCGTTTCTACAAACTCTTTCTGTGTTGTAAAACGACGGCCAGTCTTAAG
CTCGGGCCCCCTGGGCGGTTCTGATAACGAGTAATCGTTAATCCGCAAATAACGTAA
AAACCCGCTTCGGCGGGTTTTTTTATGGGGGGAGTTTAGGGAAAGAGCATTTGTCAG
AATATTTAAGGGCGCCTGTCACTTTGCTTGATATATGAGAATTATTTAACCTTATAAA
TGAGAAAAAAGCAACGCACTTTAAATAAGATACGTTGCTTTTTCGATTGATGAACAC
CTATAATTAAACTATTCATCTATTATTTATGATTTTTTGTATATACAATATTTCTAGTT
TGTTAAAGAGAATTAAGAAAATAAATCTCGAAAATAATAAAGGGAAAATCAGTTTT
TGATATCAAAATTATACATGTCAACGATAATACAAAATATAATACAAACTATAAGAT
GTTATCAGTATTTATTATGCATTTAGAATAAATTTTGTGTCGCCCTTCCGCGAAATTA
ATACGACTCACTATAGGGGAATTGTGAGCGGATAACAATTCCCCTCTAGAAATAATT
TTGTTTAACTTTTTGAGACCTTAAGGAGGTAAAACATATGAGTCCGAAAATGGTTCA
AGGTAGCGGTTGTTTTGGTCGTAAAATGGATCGTATTAGCAGCAGCAGCGGTCTGGG
TTGTAAAGTTCTGCGTgccagccctgccgcacctgcgcccgcatcacctgcggcacctgcaccttccgccccggctgcatc
tcctgccgcacccgcgcctgccagcccagctgcacctgccccaagtgcgccagcagcatcccctgccgcgcctgcccccgctagtccag
cggccccagctccatctgcaccagctgctagccctgctgcaccagctcctgcttctcccgcagccccagcgccttctgctcccgcagcctc
acctgcggccccggcaccagcatctccagcggcaccagcaccttcggcccctgctgctagcccagcagcacctgcgccagcctcacca
gctgctcccgctcctagtgccccggcggcctcgcctgctgctcctgcaccagcttcgccagcggcaccggctccttcggcgccggctgctt
caccagcagcacctgctccagcgtccccagcggcccctgctccaagtgctccggctgcatcgcctgccgctcctgctcctgcatccccag
ctgctccagcaccaagcgcacctgccgcctcaccagcggcgccagcacccgccagcccagcagcgcctgctccatccgcaccggcgG
CCTAATAAGCTTGGTTGAGGTCTCACCCCCTAGCATAACCCCTTGGGGCCTCTAAAC
GGGTCTTGAGGGGTTTTTTGCCCCTGAGACGCGTCAATCGAGTTCGTACCTAAGGGC
GACACCCCCTAATTAGCCCGGGCGAAAGGCCCAGTCTTTCGACTGAGCCTTTCGTTT
TATTTGATGCCTGGCAGTTCCCTACTCTCGCATGGGGAGTCCCCACACTACCATCGG
CGCTACGGCGTTTCACTTCTGAGTTCGGCATGGGGTCAGGTGGGACCACCGCGCTAC
TGCCGCCAGGCAAACAAGGGGTGTTATGAGCCATATTCAGGTATAAATGGGCTCGC
GATAATGTTCAGAATTGGTTAATTGGTTGTAACACTGACCCCTATTTGTTTATTTTTC
TAAATACATTCAAATATGTATCCGCTCATGAGACAATAACCCTGATAAATGCTTCAA
TAATATTGAAAAAGGAAGAATATGAGCCATATTCAACGGGAAACGTCGAGGCCGCG
ATTAAATTCCAACATGGATGCTGATTTATATGGGTATAAATGGGCTCGCGATAATGT
CGGGCAATCAGGTGCGACAATCTATCGCTTGTATGGGAAGCCCGATGCGCCAGAGT
TGTTTCTGAAACATGGCAAAGGTAGCGTTGCCAATGATGTTACAGATGAGATGGTCA
GACTAAACTGGCTGACGGAATTTATGCCACTTCCGACCATCAAGCATTTTATCCGTA
CTCCTGATGATGCATGGTTACTCACCACTGCGATCCCCGGAAAAACAGCGTTCCAGG
TATTAGAAGAATATCCTGATTCAGGTGAAAATATTGTTGATGCGCTGGCAGTGTTCC
TGCGCCGGTTGCACTCGATTCCTGTTTGTAATTGTCCTTTTAACAGCGATCGCGTATT
TCGCCTCGCTCAGGCGCAATCACGAATGAATAACGGTTTGGTTGATGCGAGTGATTT
TGATGACGAGCGTAATGGCTGGCCTGTTGAACAAGTCTGGAAAGAAATGCATAAAC
TTTTGCCATTCTCACCGGATTCAGTCGTCACTCATGGTGATTTCTCACTTGATAACCT
TATTTTTGACGAGGGGAAATTAATAGGTTGTATTGATGTTGGACGAGTCGGAATCGC
AGACCGATACCAGGATCTTGCCATCCTATGGAACTGCCTCGGTGAGTTTTCTCCTTC
ATTACAGAAACGGCTTTTTCAAAAATATGGTATTGATAATCCTGATATGAATAAATT
GCAGTTTCATTTGATGCTCGATGAGTTTTTCTAAGCGGCGCGCCATCGAATGGCGCA
AAACCTTTCGCGGTATGGCATGATAGCGCCCGGAAGAGAGTCAATTCAGGGTGGTG
AATATGAAACCAGTAACGTTATACGATGTCGCAGAGTATGCCGGTGTCTCTTATCAG
ACCGTTTCCCGCGTGGTGAACCAGGCCAGCCACGTTTCTGCGAAAACGCGGGAAAA
AGTGGAAGCGGCGATGGCGGAGCTGAATTACATTCCCAACCGCGTGGCACAACAAC
TGGCGGGCAAACAGTCGTTGCTGATTGGCGTTGCCACCTCCAGTCTGGCCCTGCACG
CGCCGTCGCAAATTGTCGCGGCGATTAAATCTCGCGCCGATCAACTGGGTGCCAGCG
TGGTGGTGTCGATGGTAGAACGAAGCGGCGTCGAAGCCTGTAAAGCGGCGGTGCAC
AATCTTCTCGCGCAACGCGTCAGTGGGCTGATCATTAACTATCCGCTGGATGACCAG
GATGCCATTGCTGTGGAAGCTGCCTGCACTAATGTTCCGGCGTTATTTCTTGATGTCT
CTGACCAGACACCCATCAACAGTATTATTTTCTCCCATGAGGACGGTACGCGACTGG
GCGTGGAGCATCTGGTCGCATTGGGTCACCAGCAAATCGCGCTGTTAGCGGGCCCAT
TAAGTTCTGTCTCGGCGCGTCTGCGTCTGGCTGGCTGGCATAAATATCTCACTCGCA
ATCAAATTCAGCCGATAGCGGAACGGGAAGGCGACTGGAGTGCCATGTCCGGTTTT
CAACAAACCATGCAAATGCTGAATGAGGGCATCGTTCCCACTGCGATGCTGGTTGCC
AACGATCAGATGGCGCTGGGCGCAATGCGCGCCATTACCGAGTCCGGGCTGCGCGT
TGGTGCGGATATCTCGGTAGTGGGATACGACGATACCGAAGATAGCTCATGTTATAT
CCCGCCGTTAACCACCATCAAACAGGATTTTCGCCTGCTGGGGCAAACCAGCGTGG
ACCGCTTGCTGCAACTCTCTCAGGGCCAGGCGGTGAAGGGCAATCAGCTGTTGCCA
GTCTCACTGGTGAAAAGAAAAACCACCCTGGCGCCCAATACGCAAACCGCCTCTCC
CCGCGCGTTGGCCGATTCATTAATGCAGCTGGCACGACAGGTTTCCCGACTGGAAAG
CGGGCAGTGACTCATGACCAAAATCCCTTAACGTGAGTTACGCGCGCGTCGTTCCAC
TGAGCGTCAGAC
(Compound 1; PAS200)
SEQ ID NO: 32
PASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPKMVQGSGCFGRKMDRISSSSGLGCKVLRRH
(PAS400)
SEQ ID NO: 33
PASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPKMVQGSGCFGRKMDRIS
SSSGLGCKVLRRH
(PAS600)
SEQ ID NO: 34
PASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPKMVQGSGCFGRKMDRISSSSGLGCKVLRRH
(PAS800)
SEQ ID NO: 35
PASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPAAPAPASPAAPAPSAPAASPAAPAPASPAAPAPSAPA
ASPAAPAPASPAAPAPSAPAASPKMVQGSGCFGRKMDRISSSSGLGCKVLRRH

### REFERENCES

Aghaabdollahian et al. (2019) Scientific Reports 9:2978.
Cataliotti et al. (2007) Trends in Cardiovascular Medicine 17:10-14.
Chen et al. (2012) J Am Coll Cardiol. 60:2305-2312.
Gengo et al. (1992) J Mol Cell Cardiol. 24:1361-1369.
Gong et al. (2016) BMJ Open 6:e008545.
Green and Sambrook (2012) Molecular Cloning: A Laboratory Manual (Fourth Edition), Cold Spring Harbor Laboratory Press, Cold Spring Harbor, N.Y.
Jefferies et al. (1996) J Med Chem 39:2331-2338.
Liu et al. (1997) J Clin Invest 99:1926-1935.
McKie et al. (2016) Eur J Heart Fail. 18:433-441.
Morath et al. (2015) Mol. Pharmaceutics 12: 1431-1442.
NCBI Reference Sequence NP_002512.1
O'Connor (2011) New England Journal of Medicine 365:32-43.
Sabbah et al. (1991) Am. J. Physiol. 260:H1379-H1384.
Sabbah et al. (2000) Circulation 102:1990-1995.
Schlapschy et al. (2013) Protein Engineering, Design & Selection 26:489-501.
Semenov et al (2011) Clin. Chem. 57:883-890.
Wan et al. (2016) J Am Coll Cardiol HF 4:539-47.
Xiong et al. (2015) PLOS ONE| DOI: 10.1371/journal.pone.0131326.
U.S. Pat. No. 5,114,923.
U.S. Pat. No. 5,674,710.
U.S. Pat. No. 6,586,396.
U.S. Pat. No. 6,974,861.
U.S. Pat. No. 7,179,790.
PCT Patent Application Publication No. WO2009156481A1.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification, including but not limited to patent publications and non-patent literature, and references cited therein, are hereby incorporated by reference. The discussion of the references herein is intended merely to summarize the assertions made by the authors and no admission is made that any reference constitutes prior art. Applicants reserve the right to challenge the accuracy and pertinence of the cited references.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

What is claimed is:

1. A modified B-type natriuretic peptide (BNP) comprising a sequence having at least 80%, at least 85%, at least 90%, at least 95%, at least 98% or 100% amino acid sequence identity to SEQ ID NO:1,
the modified BNP further comprising a covalently attached polymer comprising amino acids, wherein the polymer inhibits degradation and/or elimination of the BNP in a subject,
wherein the modified BNP retains vasorelaxant activity.

2. The modified BNP of claim 1, truncated at the C and/or N terminus of SEQ ID NO:1.

3. The modified BNP of claim 1, comprising SEQ ID NO:1.

4. The modified BNP of claim 1, wherein the polymer comprises about 200-1200 amino acids.

5. The modified BNP of claim 1, wherein the polymer comprises about 400-1200 amino acids.

6. The modified BNP of claim 1, wherein the polymer comprises about 600-1200 amino acids.

7. The modified BNP of claim 1, wherein the polymer comprises about 800-1200 amino acids.

8. The modified BNP of claim 1, wherein the polymer comprises the amino acid sequence selected from the group consisting of
(SEQ ID NO: 2)
ASPAAPAPASPAAPAPSAPA;
(SEQ ID NO: 3)
AAPASPAPAAPSAPAPAAPS;
(SEQ ID NO: 4)
APSSPSPSAPSSPSPASPSS,
(SEQ ID NO: 5)
SAPSSPSPSAPSSPSPASPS,
(SEQ ID NO: 6)
SSPSAPSPSSPASPSPSSPA,
(SEQ ID NO: 7)
AASPAAPSAPPAAASPAAPSAPPA
(SEQ ID NO: 8)
ASAAAPAAASAAASAPSAAA
(SEQ ID NO: 9)
APAAPAPAPAAPAPAPA;
(SEQ ID NO: 10)
AAPAPAPAAPAPAPAAP;
(SEQ ID NO: 11)
APPPAPPPAP,
(SEQ ID NO: 12)
PAPPPAPPPA,
(SEQ ID NO: 13)
AAPAAPAPPAAAPAAPAPPA,
(SEQ ID NO: 14)
AAAAPAAAAAAAPAAA,
and permuted or circular permuted versions or multimers(s) of these sequences as a whole or parts of these sequences.

9. The modified BNP of claim 1, wherein the polymer comprises SEQ ID NO:2 repeated at least ten times.

10. The modified BNP of claim 1, wherein the polymer is covalently bound to the N-terminus of the BNP.

11. The modified BNP of claim 1, wherein the modified BNP is P-(SEQ ID No:2)₁₀-A-hBNP(1-32) (PAS attached to N-terminal amino group), P-(SEQ ID No:2)₁₀-A-hBNP(3-32) (PAS attached to the alpha amino group of the N-Terminal lysine 3), P-(SEQ ID No:2)₁₀-A-hBNP(6-32) (PAS attached to N-terminus of glutamine 6), hBNP(1-32)-(SEQ ID No:2)₁₀-A (PAS attached to the C-terminus carboxy group), hBNP(1-30)-(SEQ ID No:2)₁₀-A (PAS attached to carboxy group of the C-Terminal arginine 30), P-(SEQ ID No:2)₂₀-A-hBNP(1-32) (PAS attached to N-terminal amino group), P-(SEQ ID No:2)₂₀-A-hBNP(3-32) (PAS attached to the alpha amino group of the N-Terminal lysine 3), P-(SEQ ID No:2)₂₀-A-hBNP(6-32) (PAS attached to N-terminus of glutamine 6), hBNP(1-32)-(SEQ ID No:2)₂₀-A (PAS attached to the C-terminus carboxy group), hBNP(1-30)-(SEQ ID No:2)₂₀-A (PAS attached to carboxy group of the C-Terminal arginine 30), P-(SEQ ID No:2)₃₀-A-hBNP(1-32) (PAS attached to N-terminal amino group), P-(SEQ ID No:2)₃₀-A-hBNP(3-32) (PAS attached to the alpha amino group of the N-Terminal lysine 3), P-(SEQ ID No:2)₃₀-A-hBNP(6-32) (PAS attached to N-terminus of glutamine 6), hBNP(1-32)-(SEQ ID No:2)₃₀-A (PAS attached to the C-terminus carboxy group), hBNP(1-30)-(SEQ ID No:2)₃₀-A (PAS attached to carboxy group of the C-Terminal arginine 30), P-(SEQ ID No:2)₄₀-A-hBNP(1-32) (PAS attached to N-terminal amino group), P-(SEQ ID No:2)₄₀-A-hBNP(3-32) (PAS attached to the alpha amino group of the N-Terminal lysine 3), P-(SEQ ID No:2)₄₀-A-hBNP(6-32) (PAS attached to N-terminus of glutamine 6), hBNP(1-32)-(SEQ ID No:2)₄₀-A (PAS attached to the C-terminus carboxy group), or hBNP(1-30)-(SEQ ID No:2)₄₀-A (PAS attached to carboxy group of the C-Terminal arginine 30).

12. The modified BNP of claim 1, wherein the modified BNP comprises SEQ ID NO:32, SEQ ID NO:33, SEQ ID NO:34, or SEQ ID NO:35.

13. The modified BNP of claim 1, wherein a half-life increase of the modified BNP, compared to the half-life of the BNP without the polymer, when administered subcutaneously into a rat, is (a) greater than 8-fold when the polymer is about 200 amino acids; (b) greater than 20-fold when the polymer is about 400 amino acids; or (c) greater than 50-fold when the polymer is about 600 amino acids or greater.

14. The modified BNP of claim 1, in a pharmaceutically acceptable carrier.

15. A nucleic acid molecule encoding the modified BNP of claim 1.

16. A nucleic acid molecule encoding any one of the modified BNPs of claim 12.

17. A vector comprising the nucleic acid molecule of claim 15.

18. A cell comprising the vector of claim 17, capable of expressing the modified BNP.

19. A method of treating a subject suffering from or diagnosed with a disease, disorder, or medical condition that can be treated with a natriuretic, diuretic or vasorelaxant, the method comprising administering to a subject in need of such treatment a therapeutically effective amount of the modified BNP of claim 14.

20. The method of claim 19, wherein the disease, disorder or medical condition is metabolic disease, pulmonary disease or heart failure.
